(19) United States
(12) Patent Application Publication
(10) Pub. No.: US 2018/0049724 A1
(43) Pub. Date: Feb. 22, 2018

(54) URINE SAMPLE DEVICE

(71) Applicant: Vasomedical, Inc., Plainview, NY (US)

(72) Inventors: Huiliang Cai, Wuxi (CN); Hong Gao, Wuxi (CN); Jianwu Zhang, Wuxi (CN); De Qing Wang, Wuxi (CN)

(21) Appl. No.: 15/682,179

(22) Filed: Aug. 21, 2017

(30) Foreign Application Priority Data
Aug. 19, 2016 (CN) 201620900506U
Aug. 19, 2016 (CN) 201620907602U
Aug. 19, 2016 (CN) 201620909321U
Aug. 19, 2016 (CN) 201620910032X
Aug. 19, 2016 (CN) 201620911101U
Aug. 22, 2016 (CN) 201620915197U
Apr. 10, 2017 (CN) 201720368708.1

(51) Int. Cl. A61B 10/00

(52) U.S. Cl. CPC A61B 10/007

(57) ABSTRACT

A urine sample device includes a funnel for receiving urine, the funnel in connection with a guide tube at a lower portion of the funnel. The urine sample device also includes a urine collection tube for collecting the urine that flows from the funnel and through the guide tube. The urine sample device further includes a guide tube fixation member located at a lower portion of the funnel and an upper portion of the guide tube along an outer periphery of the guide tube. The guide tube fixation member connected to the urine collection tube at an upper portion of the urine guide tube. The urine sample device also includes a vent located inside the guide tube fixation member.

### CROSS-REFERENCE TO RELATED APPLICATIONS

This application claims the benefit and priority of CN 201720368708.1, filed on Apr. 10, 2017; CN201620915197, filed on Aug. 22, 2016; CN 201620907602, filed on Aug. 19, 2016; CN 201620911101, filed on Aug. 19, 2016; CN 201620910032.X, filed on Aug. 19, 2016; CN 20162090321, filed on Aug. 19, 2016; and CN 201620900506, filed on Aug. 19, 2016. The entire disclosures of each of the above applications are incorporated herein by reference.

### FIELD

The present disclosure relates to a urine sampling device in the field of medical devices.

### BACKGROUND

Urine testing is a routine examination, inexpensive and can be used in a variety of diseases. But urine testing can be very troublesome, so that patients hands may be contaminated with urine, thus being very unsanitary. The market has developed several urine sampling devices. However, such structures may be associated with one or more problems, for example that structures may be irrational, cannot cope with the physical structures of men and women, require care to avoid contamination of urine sample with hands, may result in spilling, and cannot control the dose of urine collected, creating excessive urine which is not easy to deal with in the laboratory after the completion of the survey and resulting in increased possibility of environmental pollution.

### SUMMARY

A urine sample device includes a funnel for receiving urine, the funnel in connection with a guide tube at a lower portion of the funnel. The urine sample device also includes a urine collection tube for collecting the urine that flows from the funnel and through the guide tube. The urine sample device further includes a guide tube fixation member located at a lower portion of the funnel and an upper portion of the guide tube along an outer periphery of the guide tube. The guide tube fixation member connected to the urine collection tube at an upper portion of the urine guide tube. The urine sample device also includes a vent located inside the guide tube fixation member.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1 is a schematic diagram illustrating the structure according to one embodiment;

FIG. 2 is a schematic cross sectional view of the structure depicted by FIG. 1 during use according to the one embodiment;

FIG. 3 is a schematic view of the structure according to another embodiment;

FIG. 4 is a schematic cross sectional view of the structure depicted by FIG. 3 during use according to the other embodiment;

FIG. 5 is a schematic structural diagram according to another embodiment;

FIG. 6 is a schematic structural diagram according to another embodiment;

FIG. 7 is a schematic structural diagram according to another embodiment;

FIG. 8 is a sectional view according to the other embodiment of FIG. 7;

FIG. 9 is another schematic structural view of the other embodiment in use;

FIG. 10 is an enlarged cross sectional view of FIG. 9 according to the other embodiment;

FIG. 11 is a schematic structural diagram according to another embodiment;

FIG. 12 is a cross sectional view according to the other embodiment of FIG. 11;

FIG. 13 is a schematic structural diagram according to the other embodiment of FIG. 11 in use;

FIG. 14 is an enlarged cross sectional view of FIG. 13 according to the other embodiment;

FIG. 15 is a schematic structural diagram according to another embodiment;

FIG. 16 is a schematic view of the embodiment of FIG. 15 in use;

FIG. 17 is a schematic structural diagram according to another embodiment;

FIG. 18 is a schematic view of the embodiment of FIG. 17 in use;

FIG. 19 is a schematic structural view according to another embodiment;

FIG. 20 is a cross sectional view according to the one embodiment;

FIG. 21 is a schematic view according to the one embodiment in use;

FIG. 22 is a cross sectional view according to the one embodiment in use;

FIG. 23 is a schematic diagram illustrating the structure according to another embodiment;

FIG. 24 is a cross sectional view of the embodiment of FIG. 23;

FIG. 25 is a schematic diagram according to the embodiment of FIG. 23 in use;

FIG. 26 is a cross sectional view of the embodiment of FIG. 23 in use;

FIG. 27 is a schematic diagram illustrating the structure according to another embodiment;

FIG. 28 is a schematic diagram according to the one embodiment of FIG. 27 in use;

FIG. 29 is a schematic diagram according to another embodiment;

FIG. 30 is a schematic view showing the structure of a urine container according to the other embodiment;

FIG. 31 is a schematic view of a connector according to the other embodiment;

FIG. 32 is a schematic diagram illustrating the urine collecting member according to the other embodiment;

FIG. 33 is a schematic structural diagram according to another embodiment;

FIG. 34 is a schematic structural diagram according to another embodiment;

FIG. 35 shows varying schematic structural diagrams of a bowl-shaped film according to another embodiment;

FIG. 36 shows varying schematic views of a valve according to the other embodiment;

FIG. 37 is a schematic structural sectional diagram according to another embodiment;

FIG. 38 shows varying schematic diagrams of a bowl-shaped film according to another embodiment;

FIG. 39 shows varying schematic views of a valve according to the other embodiment;

FIG. 40 is a schematic structural diagram according to another embodiment; and

FIG. 41 is a schematic structural diagram according to another embodiment.

FIG. 42 is a schematic diagram of the urine receptacle contact according to another embodiment.

FIG. 43 is a schematic view of the automatic utility spool according to the other embodiment;

FIG. 44 is a schematic structural sectional view of the other embodiment;

and

FIG. 45 is a schematic structural sectional view of the other embodiment in use.

Corresponding reference numerals indicate corresponding parts throughout the several views of the drawings.

### DETAILED DESCRIPTION

The following description of technology is merely exemplary in nature of the subject matter, manufacture and use of one or more inventions, and is not intended to limit the scope, application, or uses of any specific invention claimed in this application or in such other applications as may be filed claiming priority to this application, or patents issuing therefrom. A non-limiting discussion of terms and phrases intended to aid understanding of the present technology is provided at the end of this Detailed Description. Exemplary embodiments will now be described more fully with reference to the accompanying drawings.

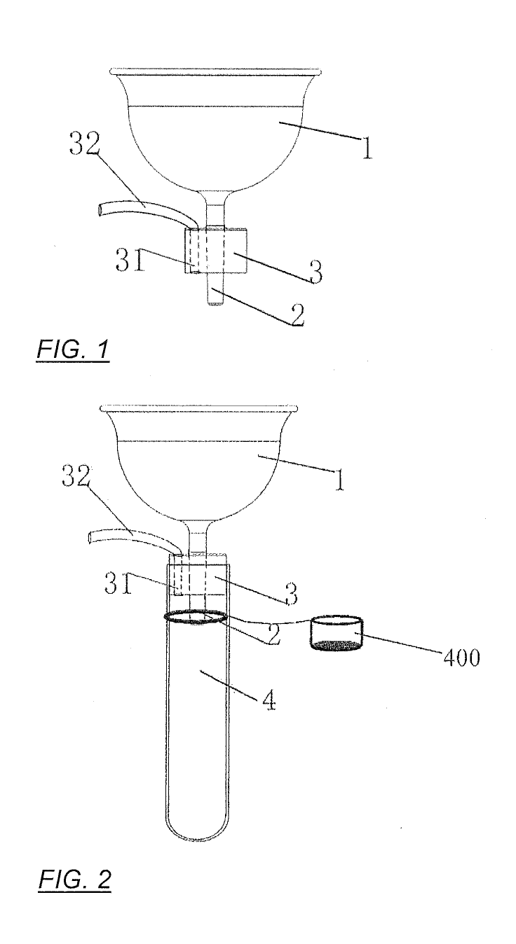

Referring to FIGS. 1 and 2, a self-ventilating urine sampling device includes a bell-shaped mouth section 1 for holding urine (and may also be referred to as a bell, urine sampling funnel, a urine sampling bag, or a urinary funnel), a guide tube 2 (also known as a urine gathering tube) provided at the lower end of the bell-shaped mouth section 1, a guide tube fixation plug 3 (which may also be referred to as a guide tube fixation ventilation mechanism or a guide tube fixation member) located on upper part of the outer diameter of the guide tube 2, and at the upper end and within the inner diameter of urine collection tube 4, and a vent 31 located within the guide tube fixation plug 3.

When receiving the urine, the bell-shaped mouth section 1 has its bell opening facing up and directly facing the opening of patient's urethra, so when the urine flows out of the urethra, the external shape of the bell opening reduces splashing of urine, and avoids urine spill and leakage that dirties the patient's fingers. After the urine flows into bell-shaped mouth section 1's cavity, it builds up towards the bottom of the bell-shaped mouth section 1, through the lumen of the guide tube 2 and flows into the urine collection tube 4.

The outer diameter of the guide tube fixation plug 3 meets with the inner diameter of the upper part of the urine collection tube 4, allowing for a tight seal between the interior wall of the urine collection tube 4 and the outside wall of the guide tube fixation plug 3 after the fixing the urine collection tube 4 to the guide tube fixation plug 3. The effect is: the urine collection tube 4 that is connected with the guide tube fixation plug 3 can be temporarily fixed to avoid falling; when the urine flows into the urine collection tube 4, the air inside the urine collection tube may be discharged through the vent 31 inside the guide tube fixation plug 3; when urine collection tube 4 is filled with urine, extra urine can be discharged through the vent 31; the tight sealing contact between the outer wall of the guide tube fixation plug 3 and the inner wall of the urine collection tube 4 prevents excess urine from leaking through in between the outer wall of the guide tube fixation plug 3 and the inner wall of the urine collection tube 4 to contaminate the outer wall of the urine collection tube, thus preventing an operator's hands from getting dirty.

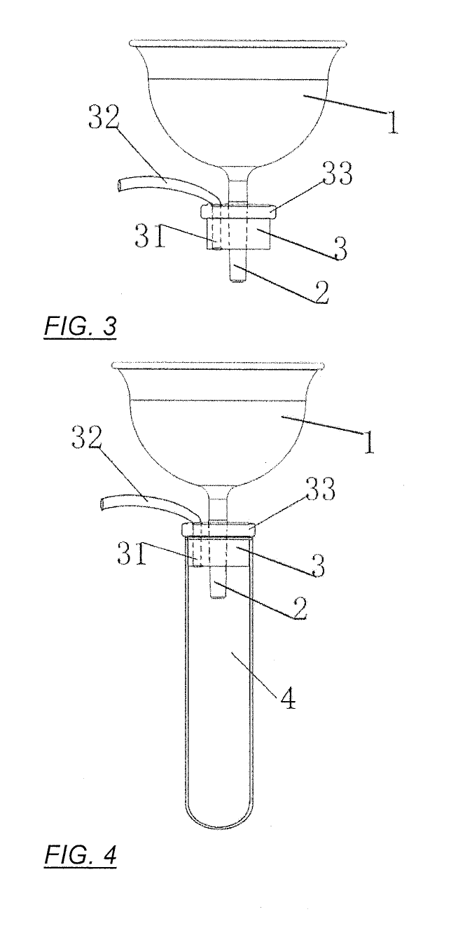

Referring to FIGS. 3-6, the upper end of guide tube fixation plug 3 includes a spacer 33, the spacer 33 can be taken on the upper edge of the stopper 4 urinary catheters. The specific embodiment, the inner diameter of the lower part of the guide tube fixation plug 3 and urine collection tube 4 matching can take into upper interior urine collection tube 4 and sealing contact with the inner wall of urine collection tube 4, urine collection tube 4 is temporarily fixed plug on the guide tube fixation plug 3 and will not fall off. The stopper 33 is greater than the outer diameter of urine collection tube 4 that can take urine collection tube 4 placed on the guide tube fixation plug 3 stopper 33, when the upper urine collection tube 4 to take the opening edge of the top support to the stopper 33 so, sleeved aborted. The role of the stopper 33 is: to ensure that urinary catheters and urine collection tube 4 sets of 3 will not set too tight, that they can be removed when inconvenient; urine collection tube 4 may be pulled out of the catheter fixing plug 3; that the operator can limit stopper 33 as part of a handheld, and is easily detachable.

As shown in FIGS. 1-6, the exhaust hole 31 is provided connected to the exhaust pipe 32, the exhaust pipe 32 and sealing the exhaust hole 31 communicating. Sealing of the exhaust hole 31 may be provided to connect the exhaust pipe 32 when the urine in urine collection tube 44 overflows through the vent hole 31, and then discharged from the exhaust pipe 32. In extending the guiding role of the exhaust pipe 32 to take away from the urine from urine collection tube 4, the device avoids contamination of urine on outside of urine collection tube 4, and avoids dirtying the hands of the operator while handling the urine collection tube 4.

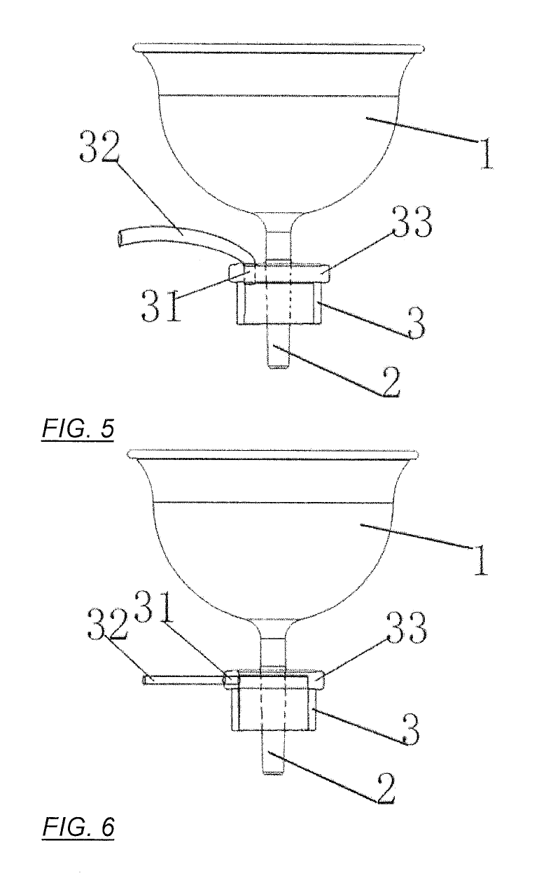

The vent 31 provided in the fixed portion of the guide tube fixation plug 3 is located near the bell 1 side. As shown in FIGS. 1-5, the vent pipe 31 is fixed up and down through the long axis guide tube fixation plug 3 set. Guide tube fixation plug 3 can be set to a fixed entity of the plug body, but also can be set to cover the upper part has an empty cylindrical plug body, in there is no big difference when using. FIG. 6 vent pipe 31 extends through the lumen side of the fixed side wall guide tube fixation plug 3, when the guide tube fixation plug 3 must be set to the upper portion of the closure has empty cylindrical plug body, otherwise vent tube 31 is difficult to take in fluid communication with the urine collection tube 4.

It is noted that, when the urine is extracted, the exhaust hole 31 and the exhaust pipe 32 may trace residual urine, when the detachable fixing guide tube fixation plug 3 disposed in the catheter exterior wall of the urine collection tube 4, the fixed guide tube fixation plug 3 is sufficient space vacated remaining trace urine, residual urine is taken to avoid contamination of the urine collection tube 4, but also to avoid contamination of the urine sample operator.

Also provided is a cap 400 for sealing the urine inside the urine collection tube 4. After urine sampling collection is finished, the upper structure including the flared portion 1, the guide tube 2, the guide tube fixation plug 3 including the vent 31 and ventilation tube 32 is removed, and the cap 400 is placed over the upper part of the urine collection tube 4, thus securely and more permanently sealing the urine inside the urine collection tube 4 for future use.

The above allows for a urine sampling from the discharge member, in which flared portion 1 for containing the urine, urine collection tube 4 taken through guide tube fixation plug 3 is temporarily fixed fixing plug, into the urine through the guide tube 2 take urine collection tube 4, the gas from the urine collection tube 4 is discharged through the exhaust hole 32 and the exhaust pipe 31, to avoid contamination of medical patients. The above provides for a simple structure that is low in cost, safe and reliable.

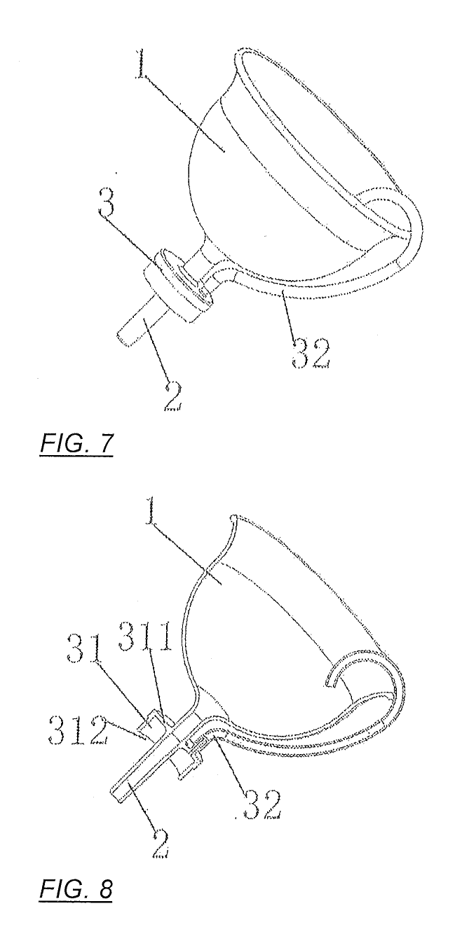

In another embodiment, as shown by FIGS. 7, 8, 11, and 12, a self-discharge funnel urine samples, including a bell shaped mouth section 1 for holding urine, provided at the lower end of the bell cavity and the guide tube 2 and a flared portion provided in the guide tube communicating with bell shaped mouth section 1 2 into the outer wall of the insert taken for urinary catheters 4 and can take the urinary catheter fixing mechanism exhaust gas discharged from conduit 4 within three. The exhaust duct fixing mechanism and the lower part 3 is set to take the upper end of the outer wall of urinary catheters 4 matches catheter lock chamber 31, the catheter lock chamber 31 near the side of the bell section 1 of the sealed end 311, cavity 31 away from the catheter fixing bell shaped mouth section 1 an open end 312 side. Lumen catheter fixed in the sealed end 311 side 31 and catheter fixing cavity 31 communicates with the exhaust pipe 32.

When receiving the urine, the bell shaped mouth section 1, reduces splashing of urine, urine spill and avoids spilling on to the patient's hands. Urine collected into the bell portion of the cavity, builds up to the bottom of the bell section 1 through the lumen of the guide tube 2 and flows into the taking of urine catheter lumen 4.

The fixed duct cavity 31 and the upper urinary catheter outer wall 4 matches that take four sets of urinary catheters set sealing contact with the inner wall of the cavity 31 of the fixed duct 31 of the catheter after taking lock chamber and the outer wall of the upper urinary catheter 4. The effect is: sleeve set into the fixed lumen catheter 31 to take temporary urinary catheters 4 can be fixed to avoid falling; when taken into the urine within the urinary catheter 4, take air through the urine guide tube 2 and take urinary catheter conduit 4 within 4 after discharge gap lumen catheter into the lock chamber 31 into the air lock chamber 31 within the catheter can be discharged through the exhaust pipe and pipe fixing cavity 31 in communication 32; when taken within four urinary catheters filled with urine, the guide take the tube 2 into the urine excess urinary catheters 4 can be discharged through the exhaust pipe 32.

FIGS. 7-10, the exhaust pipe 32 4 disposed in the cavity 31 of fixed duct seal the end of the end portion 311. This pollution takes urinary catheter structure 4 which are appropriate, urine collection tube 4 of the sealing membrane 5 is provided with a fissure, crack sealing film 5 to the central depression of the bowl-like structure, provided with a plurality of recessed bottom crack. If the force is consistent with the direction of the recess, crack opening, the guide tube 2 can smoothly enter the urine collection tube 4 are taken. Reverse by a smaller force, shrink cracks, fissures closed so enhanced, taking the urine of gas or liquid within the conduit 4 does not flow out. But the force is large solids can smoothly access.

After, the means to take urine collection tube 4 into the catheter end portion 31 of the lock chamber, an outer wall and an inner wall 31 takes urinary urine collection tube 4 is fixed in sealing contact, but the fissure sealing film 5 of the bowl-shaped recess formed basin body structure space, space is on the basin body sealing end 311 of the end portion of the bowl to take up space communication urine collection tube 4 and an exhaust pipe 32.

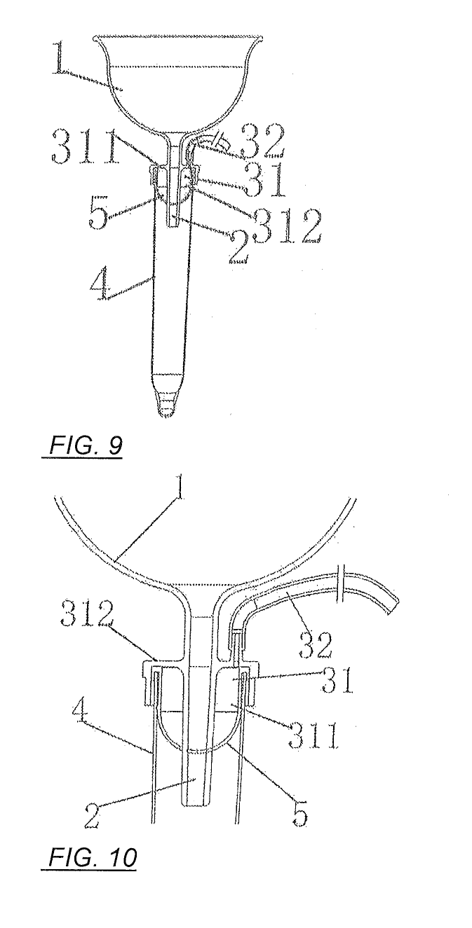

Referring to FIGS. 9 and 10, will take before using urine collection tube 4 through cracks fissure sealing film 5 is set into the outer wall of the guide tube 2 and take home four sets of urinary catheters and catheter fixing the insert into the cavity 31 within which to take the top edge of urine collection tube 4 holding the ends of the catheter lock chamber 31 sealed ends 311. Then the bell section 1 is on the urethra urination, urine in the trumpet section 1l protect concentrated at the bottom into the guide tube 2, and 2 through the guide tube into the lumen of the catheter within the urinary take 4. At the same time, take the urine 4 pipe gas through fissures in the membrane seal the gap between 2 and 5 of the guide tube is discharged into the basin body space fissure sealing film 5, and then with the bowl-shaped space communicating the exhaust pipe 32 is discharged.

When taken within four urine filled urinary catheters, urinary catheters to stop urinating remove take 4 complete urine sample. When the patient is not found in time, proceed to take urine urinary catheter 4, the excess urine along the gas discharge passage by the exhaust pipe 32, inside the bell section 1 avoid the accumulation of excessive urine overflow easily contaminated patients hand.

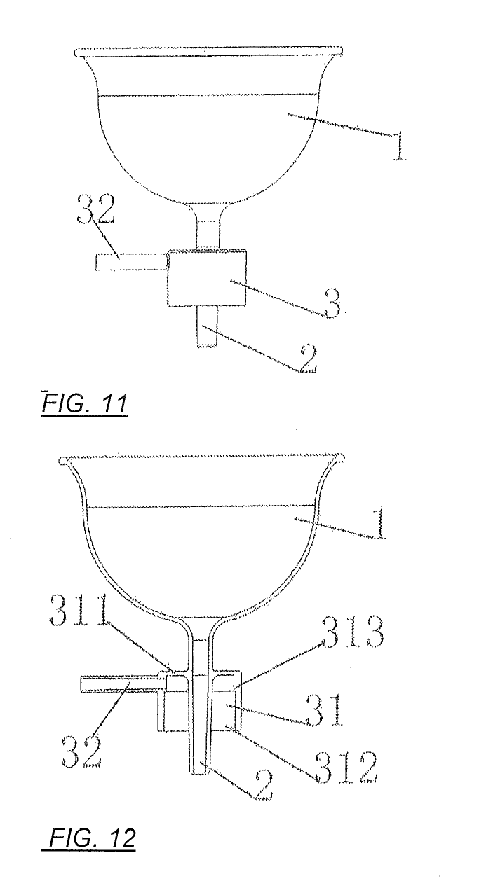

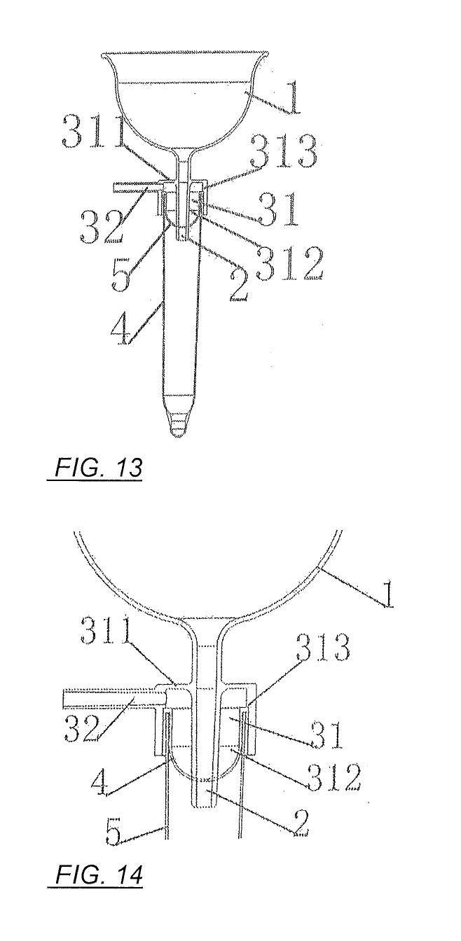

Referring to FIGS. 11 and 12, and as shown in FIG. 7 the exhaust pipe 32 is disposed near the sealed end of the conduit 311 fixed to the end portion of the side wall 8 cavity 31. At the same time, the cavity 31 of fixed duct lumen sealed end 311 side end of the stopper 313, the stopper 313 is not lower than the limit point of the exhaust gas inlet pipe 32.

If setting stopper 313, take 4 urinary catheters inserted catheter lock chamber 31 easy to make too deep within the exhaust pipe 32 of the gas inlet pipe 4 is taken on the edge of the outer wall of the urinary blockage, would not achieve the effect of exhaust, leading to urinary failure. If we take 4 urinary catheters inserted catheter lock chamber 31 is too shallow, poor urinary catheter 4 is secured to take effect, easy prolapse; 31 and the inner wall of the outer wall 4 to take urinary catheters and catheter lock chamber is also prone to poor sealing contact effect occurs in urine was taken by urinary catheter slit 431 and the inner wall of the outer wall of the cavity between the catheter fixing leaking, leading to contamination of urine taken 4 outer urinary catheters, patient hands contaminated urine.

It is set so that the stopper 313 is fixed to take the urine catheter lumen catheter 31 to achieve the best depth, both to ensure the exhaust gas inlet pipe 32 will not be taken on the edge of the outer wall of the urinary catheter 4 jam. But also to ensure the fixed effects take urinary catheters 4, it avoids taking urinary catheters 4 off. But also to ensure the take urinary catheters and catheter fixing the outer wall 4 wall sealing cavity 31, to avoid contamination of urine taken urinary catheters 4 outer wall.

It need to be reminded that, when the end of the urine sample, taken at the upper urinary levels of 32 and an exhaust pipe conduit 4 fissure sealing film 5 may accumulate a small amount of urine. But with the guide pipe 2 taken from urinary catheters pulled within four, taking the urine will form a negative pressure within the catheter 4, the above accumulated urine adsorption housed in urinary catheters 4 are taken. In the specific embodiment, the guide tube 2 into the substance of the volumetric capacity to take 4 urinary catheters should be greater than the volume of accumulated urine in order to avoid a small amount of urine leakage, to avoid contamination of patients. Meanwhile, in order to ensure the existence of a gap between the use of crack sealing film 2 and the guide tube 5, fissure sealing film 5 should not be too small, and the outer diameter of the guide tube 2 should not be too large. If the urine is not set up to take fissure sealing inner catheter 4 film 5, the utility model can also be used normally, but the end of the urine sample, taken unplug urine collection tube 4 and inspection process required to be very careful to avoid contamination of urine splashing operator hand.

The above provides a urine sample from the exhaust funnel, its bell unit 1 received for holding urine, take temporarily urinary catheter 431 fixed to the lower fixed bell 1 through the catheter lumen, the urine through the guide tube 2 take the urine into the urine collection tube 4, take the gas in the urine collection tube 4 gas through the guide tube 2 and 4 take lumen catheter urinary void discharged into the vent 31 and ventilation tube 32 is discharged through the exhaust pipe. When there is too much urine, excess urine will overflow through the ventilation tube 32, to avoid the risk of contamination of health care.

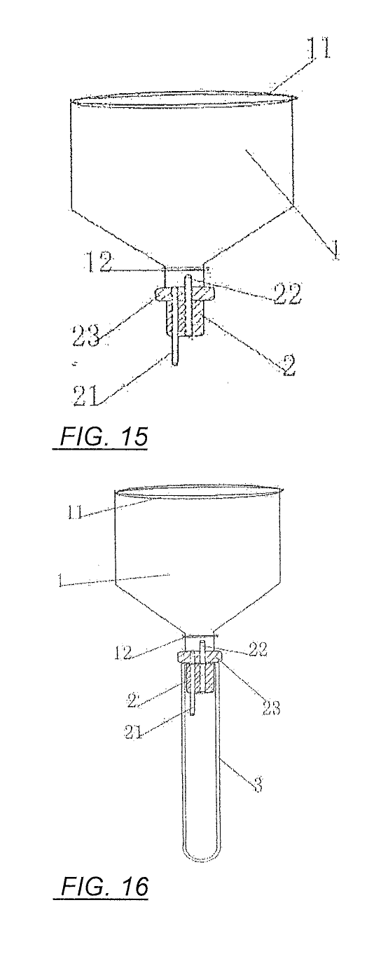

In yet another embodiment, as shown in FIGS. 15-18, a urine sampling bag includes a urine sampling funnel in the form of a urine sampling bag 1 for collecting urine, and urine collection tube 4 can be fixed at its upper portion to the lower portion of the sampling bag 1 at the guide tube in the form of a guide tube fixation member 2. The catheter is fixed within a lumen of the guide tube 2 and the sampling bag 21 and the interior cavity I ventilation tube 22.

Before urine collection, a urine collection tube 4 is tightly fixed and sealed to a guide tube in the form of a fixation member 2. Both a guide tube 21 and a ventilation tube 22 are located in the inside of the fixation member 2 and the sealing member is fixedly connected to the fixed conduit, the urine guide tube of the catheter taken through the lumen of the urine collection tube 4 provided on the fixed body 221 and the discharge hole 22 and the sampling bag I are in communication. Sampling bags placed in front of the I urethra when the urine sample extraction, open the bag, the bag avoid physical contamination of urine splashing. Agglomeration into the urine in the sampling bag 1 at the bottom of the sampling bag through the guide tube 21 into the conduit 2 fixed urine collection tube 4 to take the urine catheter lumen; At the same time, taking urinary catheters gas in the urine collection tube 4 through the conduit 2 fixed body in the ventilation tube 22 is discharged, thereby completing the taking of urine.

To ensure the smooth extraction of urine, the length of the ventilation tube 22 located at the guide tube fixation member 2 of the sampling bag 1 is the same length as that of the guide tube 21, so when the urine flows into the bottom of the bag, the inlet of the guide tube 21 is situated at a position lower than that of the inlet of the ventilation tube 22, urine can first flow to the first inlet guide tube 21 into the urine collection tube 4, preventing the clogging of the ventilation tube 22, making gas discharge from the urine collection tube 4 poor.

In order to ensure that the gas inside the urine collection tube 4 is discharged freely, the length of the guide 21 on the side of the catheter away from the fixed sampling bag body than on the same side of the vent hole 22 long tube. urine collection tube 4 taken in the urinary catheters, the air located above the urine, an exhaust vent port portion 22 than the guide tube 21 higher, to facilitate the smooth discharge of air. When the urinary catheter takes the urine level rises urine collection tube 4 blocked the vent exhaust port 22, the residual gas in the conduit urinary collection tube 4 to stop taking the urine discharged, taking the end of the urine.

The sampling bag mouth portion 1 is provided with an elastic ring 11 that may expand the end of the sampling bag mouth portion 1. Under the action of elastic ring 11, a sampling port portion of the bag I can remain open, to facilitate the extraction patient urine.

At the end of the extraction urine, urinary catheters need to be taken urinary collection tube 4 detachment from the fixed body 2 of the catheter, urinary catheter in order to avoid detachment during the fixed body 21 on the outflow guide tube contamination or bottom surface of the second operation, the sampling bottom of the bag 1 can be disposed at the switch 12 inlet guide pipe 21. Thus, detachment urinary catheters to take off before the switch 12, thereby eliminating urine contamination resulting from detachment of the guide tube fixation member 2 from the urine collection tube 4.

The upper portion of the stopper body 2 of the catheter body 23 can be fixed urinary catheter urinary collection tube 4 for taking the stopper portion. The urinary catheter takes the catheter urinary collection tube 4 is fitted on the fixed body 2, if there is no limiting effect of the stopper body 23 may cause urinary catheter urinary collection tube 4 is fitted to take too deep too tight, resulting in difficulties in detachment. The stopper member 23 is provided to avoid such concerns, but also to avoid operator assembly too shallow, resulting in taking urinary catheters and catheter urinary collection tube 4 is temporarily fixed fixing member 2 is too loose, the urine taken in the operation urinary collection tube 4 to take urinary catheters accidental removal.

The guide tube fixation member 2 includes a catheter lumen and a matching outer wall of the conduit urinary collection tube 4 taking urinary catheter and an outer sleeve fixed with the lumen of the catheter taken urinary catheters urinary collection tube 4 fixed matching plug. As shown in FIGS. 15 and 16, the catheter body 2 is fixed to the catheter anchor plug. When using the catheter fixing plug taken into urinary catheters can lumen portion urinary collection tube 4, while the stopper 23 is set larger than the projection of the plug body, the plug is fixed into the catheter to avoid taking too deep urinary catheter urinary collection tube 4.

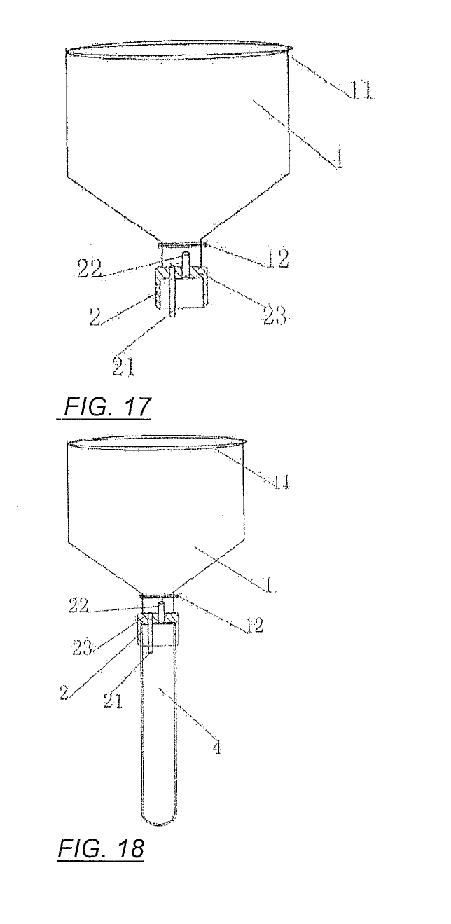

As shown in FIGS. 17 and 18, the guide tube fixation member 2 is fixed to the conduit sleeve. When using sleeve disposed in the sleeve taken urinary catheters urinary collection tube 4 to the outer wall portion while the stopper member 23 is provided to cover the upper end of the cylinder, to avoid taking too deep urinary catheters within the catheter urinary collection tube 4 into the fixed sleeve.

In order to reduce costs, the sampling bag I is preferably a film bag, which is collapsible and compressed before use to reduce transport costs.

The above provides a urine sampling bag sampling bag stretched elastic ring 11 facilitates the extraction of urine I mouth portion, the fixed body 2 to facilitate the catheter taken urinary catheters urinary collection tube 4 is temporarily fixed, the urine guide pipe 21 guide the catheter into the urinary taken urinary collection tube 4, taken through the urinary catheter urinary collection tube 4 into the gas exhaust pipe 22 is discharged, the switch 12 to close the bottom of the bag takes after the completion of taking of urine, urinary catheters urinary collection tube 4 to remove taken. Structure is simple, inexpensive and easy to use; and cost, pollution may be small, making the structure easy to promote for use.

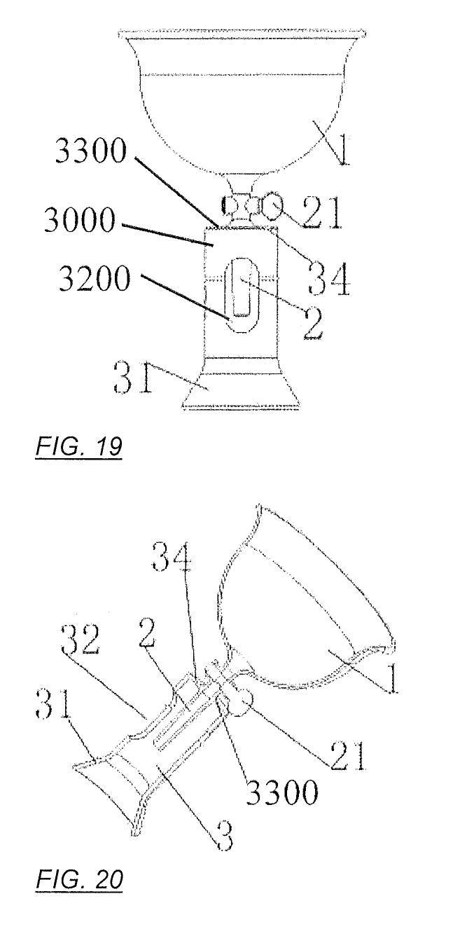

In yet another embodiment, as shown in FIGS. 19, 20, and 22, a urine sampling funnel with a manually-operated valve, including an inverted and flared trumpet bell-shaped section 1 for receiving urine, a guide tube 2 provided at the lower end of the bell section 1, and a urine collection tube fixation sleeve 3000 located on the guide tube 2 and the mouth portion is provided in the communication I 2 for fixing the guide tube 4 is taken to take urine collection tube fixation sleeve 3000. The guide tube 2 is provided with a manual valve 211, the operator can control the control valve 211 opening and closing the guide tube 2.

When receiving the urine, the upwardly flared bell mouth portion I, n patient urethra opening, the bell mouth shape to reduce urine after urine splashing out of the urethra, and to avoid spilling urine spill pollution patient's finger. Urine entering the bell mouth 1, I accumulated to the bottom portion of the bell mouth 1, flows through the guide tube 2 into the lumen of the catheter 4 lumen of urine taken.

The urinary catheter takes the fixed urine collection tube fixation sleeve 3000 and the upper end of the outer wall 4 to take matching urinary catheters, urinary catheters make take 4 sets of urine taken disposed in the inner wall of the upper end 4 of the catheter fixing an upper portion of the urine collection tube fixation sleeve 3000 fixed sleeve lumen of the urine collection tube fixation sleeve 3000 and take urinary catheters sealing contact with the outer wall.

The structure described above allows for the following: urinary catheters sleeved taken into the conduit sleeve 4 can be fixed to temporary fixing of urine collection tube fixation sleeve 3000 to avoid falling off; when taking urine from the urine into the conduit 4, the air taken through the urinary catheter guide tube 42 after taking urinary catheters and the gap is discharged into the lumen 4 taken urinary catheters fixing sleeve at the top of the cavity urine collection tube fixation sleeve 3000 into the urine taken in the air duct 3 and is fixed via the sleeve can be fixed to the conduit urine collection tube fixation sleeve 3000 taken urinary discharge.

In the guide tube 2 is provided a manual valve 211, the operator can control the control valve 211 open and close the guide tube 2, the amount of urine the urine can take autonomous control in the conduit 4 entering.

Before use, the manual valve 211 the guide tube 2 in the closed state, take 4 sets of urinary catheters placed in the upper chamber to take a urine collection tube fixation sleeve 3000, the said flared portion I flare upwardly facing patient's urethra receiving mouth opening of the urine, the urine collected in the cavity of the bell mouth portion 1.

The flared portion of said reservoir urine samples were placed in front of I, manual valve 211, the guide tube 2 is open, the bell mouth portion I 2 into the urine reservoir cavity taken by the lower guide pipe 4 urinary catheter. Meanwhile, taking into the gas conduit 4 via the urine guide tube 2 and the outer wall of the catheter taken urinary clearance of the inner wall 4 is discharged. If we take urine from the conduit 4 is provided fissure sealing film 5, the urinary catheter taken through the gap 4 into the gas discharge pipe 2 and the outer wall of the fissure sealing film 5 a guide slit diaphragm.

When the urine flowing into the urine collection tube 4 reaches, manual valve 211, the guide tube 2 is closed, to pull take urinary catheters 4, and the internal residual perhaps urine excess present invention new discarded, complete urine sample. If the fear of urine splashing of urine taken in the conduit 4, the one-way membrane may be taken urinary catheter 4, such as fissure sealing film 5 and the like.

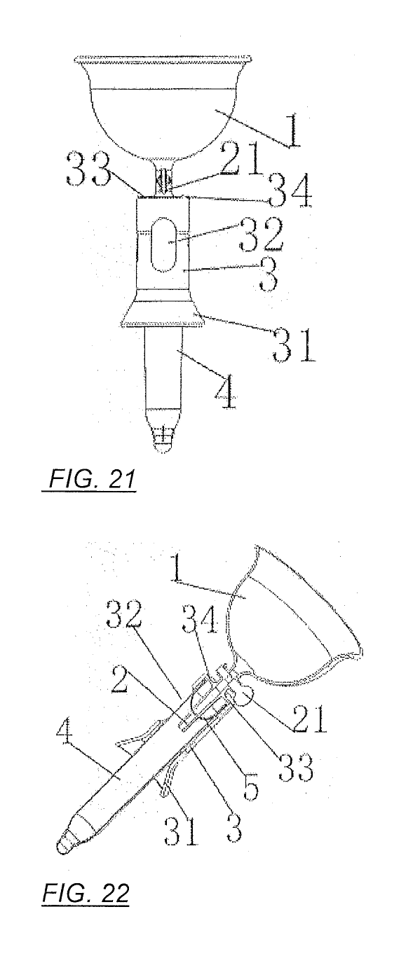

Referring to FIGS. 19-22, the urinary catheter takes urine collection tube fixation sleeve 3000 and the upper end of the outer wall 4 to take matching urinary catheters, urinary catheters to take away from the urine collection tube fixation sleeve 3000 a flare portion I a tapered mouth 3100 side. The urine collection tube fixation sleeve 3000 and the upper end of the outer wall 4 taken urinary catheters match-enabled set to take 4 sets of urinary catheters into the urine collection tube fixation sleeve 3000 is temporarily fixed when the fixing sleeve can take urine, urinary duct 4 to avoid taking off when the urine sampling, resulting in the flow of urine pollute the environment or operator. In the urine collection tube fixation sleeve 3000 taken away from the side where the tapered flare portion I tapered mouth 3100 side is located, to facilitate the taking of urine into the conduit 4 opposing sets of upper urine collection tube fixation sleeve 3000, to reduce the cumbersome alignment operation.

The urine collection tube fixation sleeve 3000 taken upper portion 4 takes the observation window 3200 urinary catheters. By observing the sighting tube can urine capacity observed in the conduit 4 entering the taking of urine, urine within the urine taken to avoid entering the excessive overflow conduit 4, the flow of urine to avoid contamination to the operator

The urinary catheter takes a urine collection tube fixation sleeve 3000 is provided near the side of the bell mouth portion I conduit 4 can stop the space restriction block 3300 pairs taking urine.

The space restriction block 3300 to take into taking urinary catheters 4 urinary catheter fixing depth within the urine collection tube fixation sleeve 3000 play the role of positioning, so that the depth of the system to achieve the best effect, taking into urinary catheter taken within the urine collection tube fixation sleeve 3000 4 urinary catheters function sufficiently temporary fixation, to avoid too loose or too tight to avoid urinary catheters 4 take off or urine sampling after completion of urinary catheters is difficult to take the 4 coming out from the take-urine collection tube fixation sleeve 3000.

The urine collection tube fixation sleeve 3000 is provided in communication with the vent hole 3400. Vent hole 3400 provided in the catheter so that the urine flows into the urine collection tube 4, the gas in the conduit 4 can smooth discharge of urine taken to avoid the taking of urine gas in the conduit 4 cannot be discharged due to the failure urine sampling.

The above described structure connected to the bell mouth portion 1 prevent urine splash, with the space restriction block 3300 assisting in the taking of urine can be temporarily fixed to the conduit 4 urine collection tube fixation sleeve 3000, the manual valve 211 can controlling the guide tube 2 is open and closed to control the capacity of the urine sample, the vent hole 3400 to ensure that the gas in the conduit 4 taken smooth discharge of urine, the sampling process to ensure clean and simple, convenient and reliable, low cost, practical.

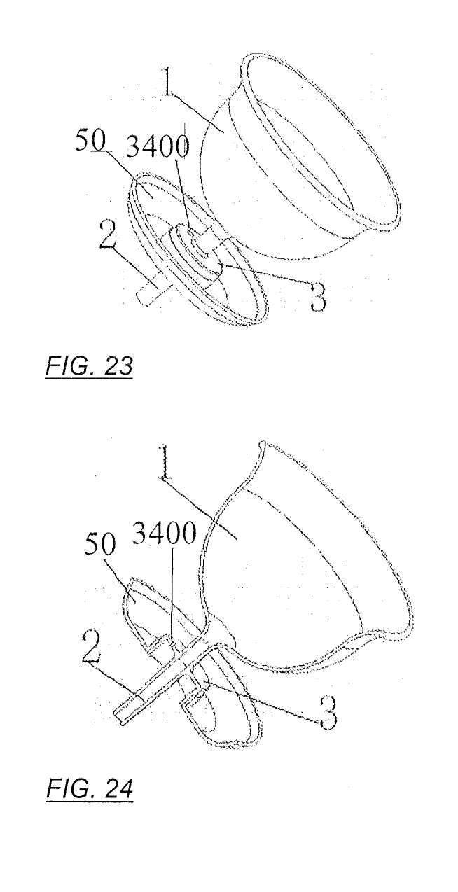

In yet another embodiment, and referring to FIGS. 23, 24, 27, and 28, an anti-overflow tray 50 is connected to the bell portion 1 for containing the urine leaking out from the inverted bell mouth portion 1, lumen and a flare bell mouth portion provided at the lower end of the guide tube communicating with I 2, is provided for the guide tube fixing member 2 fixing the urine collection tube 4 taking 3 and urinary catheters for containing urine excess spill plate 50. The conduit 3 is provided for the fixing member escape hole is discharged into the gas duct urine collection tube 4 to take the urine and excess urine vent hole 3400, the bottom spill plate 50 of the vent hole 3400 below the overflow, said vent hole 3400 provided inside the spill plate 50.

When receiving the urine, the upwardly flared bell mouth portion 1, n patient urethra opening, the bell mouth shape to reduce urine after urine splashing out of the urethra, and to avoid spilling urine spill pollution patient's finger. Urine entering the urine sampling funnel 1 is accumulated to the bottom portion of the bell mouth, flows through the guide tube 2 into the lumen of the urine collection tube 4 taking the urine lumen.

The guide tube fixation member 3 and the urine collection tube 4 can be temporarily fixed, fixing the guide tube fixation member 3 and urine collection tube 4 taken urinary catheters upper sealing contact with the adjacent side wall. Guide tube fixation member 3 and urine collection tube 4 are temporarily fixed, urinary process must avoid taking hand fixed and reduced the urine taken during the taking off urine collection tube 4. When taken into the urine when the urine overflowing the inner conduit urine collection tube 4, to avoid a gap between 3 and urine collection tube 4 taken urinary catheters excess urine flows out through conduit fastener, urine sample contamination operator's hand portion.

The fixing member 3 and the conduit to take the upper end of the adjacent side walls urine collection tube 4, when urine enters the urine collection tube 4 taking the urine, the urine taken through the vent hole 3400 exhaust conduit in the urine collection tube 4 is fixed to the upper portion 3; when urine enters the overflowing when taking urine from the urine collection tube 4, the excess can be discharged through the overflow orifice urine catheter fixing member 3 of the vent hole 3400 upper portion. The escape hole 4 is provided in the spill spill plate 50 and spill plate 50 is higher than the spill tray, allows excess urine to flow into the vent hole 3400 and spill plate 50 accumulated in the spill tray.

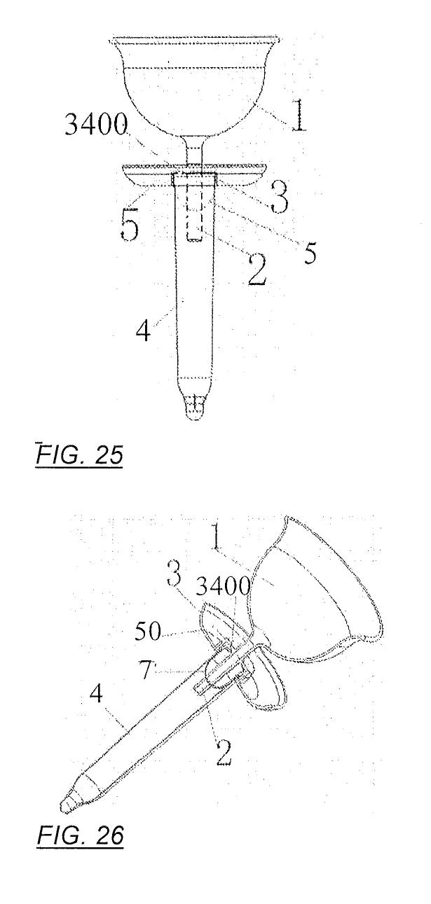

As shown in FIGS. 25, 26, the catheter 3 includes a fixing member fixed to the catheter sheath 31, the inner diameter of the catheter fixing sleeve taken urinary catheters 31 matching the outer diameter of the upper portion urine collection tube 4. After the catheter 31 is provided a lower fixing sleeve, and an inner diameter matching the outer diameter urine collection tube 4 taking the urinary cavity, taking the counter into the urine collection tube 4 is fixed inside of the catheter sheath 31, the inner wall urine collection tube 4 will take the upper urinary catheter 31 and the outer wall of the catheter anchor sleeve can upper sealing contact.

When the catheter fixing member 3 fixed to the catheter sleeve 31, is preferably provided in an observation window urine catheter fixing sleeve sidewall 31 can be taken into the visual inspection of the amount of urine in the urine collection tube 4. 31 is preferably provided at a lower portion of a bell mouth fixed sleeve catheter, the urine collection tube 4 to facilitate the taking of urine into the sleeve 31 facing the catheter anchor sleeve.

In FIGS. 25 and 26, the upper end of the inner urine collection tube 4 taking the urine cavity 5 is provided fissure sealing film, the urine the urine can be taken to avoid the contamination of the urine collection tube 4 spill hands after sampling. But the fracture should be noted that the sealing film 5 is provided to ensure that the center longitudinal fracture fissure 5 seal film, should ensure that the guide tube when the guide tube 2 via the center fracture fissure sealing membrane 5 into the urine collection tube 4 taken urinary catheters outer wall fissure seal member 5 and the central fissure sealing film 2 fracture edge should be a sufficient gap, so that the urine collection tube 4 taken urinary catheter lower energy is discharged through this gap, and then discharged through the vent holes 3400; and urinary catheters take urine overflowing urine collection tube 4, the excess urine can after this discharge gap, and then discharged and collected in the spill plate 50 through the vent hole 3400.

If the urine cavity is not provided within the upper end of the conduit sealing film fracture 6 fissure seal membrane 5 does not affect the urine sample, a urine sample just after the operator moves the urine sample be careful to avoid taking urine from the upper opening of the urine collection tube 4.

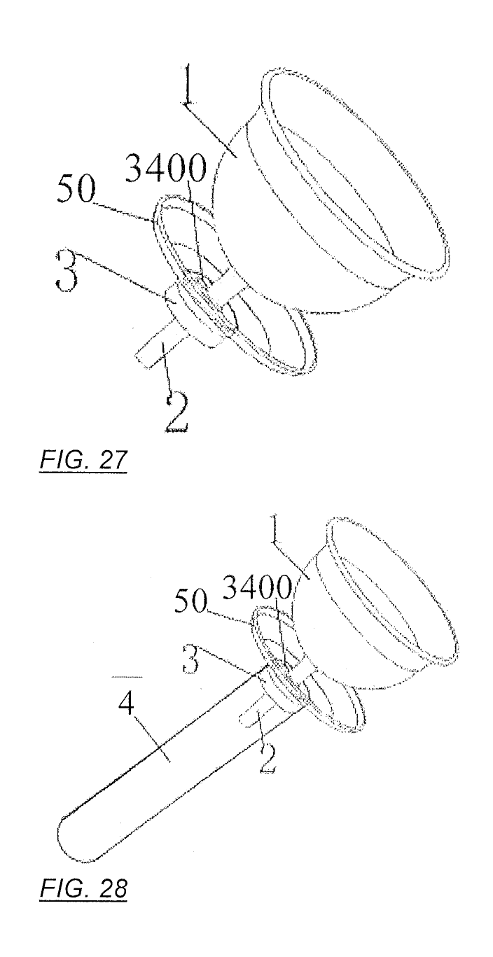

As shown in FIGS. 27 and 28, the upper inner diameter catheter fixing member 3 fixed matching plug 32 includes a catheter, the outer diameter of the catheter fixing plug taken urinary catheters 32 urine collection tube 4. The lower portion of the outer wall of the catheter fixing plug 32 should match the inner diameter of the upper conduit urine collection tube 4 taking in urine, and can be sealed in contact. When the urine sample, the urine is I 2 flows into the bell mouth portion of the guide tube when taking urine from the urine collection tube 4, the air taken through the vent holes 3400 The urine discharge urine collection tube 4. When urine overflowing, excess urine is discharged through the vent holes 3400 and accumulated in the spill plate 50.

When the catheter fixing member 3 fixed to the conduit plug 32, functions as an upper stopper 32 fixed to the bottom of the conduit spill plate 50 of the spill tray limiting effect can be avoided catheter fixing plug 32 is embedded into the urine collection tube 4 taken urinary catheters depth too deep, taken at the end of the urine collection tube 4 urinary catheters difficult to remove.

There should be sufficient volume of the spill plate 50 accommodating excess urine, but also patients with normal sampling, control the discharge of urine, to avoid excessive urine volume than the spill plate 50 and spilled.

With the above-described structure: the bell mouth portion 1 prevents urine from contacting splashing of urine taken to ensure that vent holes 3400 in the gas discharged smoothly urine collection tube 4, to avoid contamination of the spill plate 50 when taking urine overflowing urinary urine collection tube 4 of the outer wall, to ensure that the sampling process is simple and clean, convenient and reliable, low cost, practical.

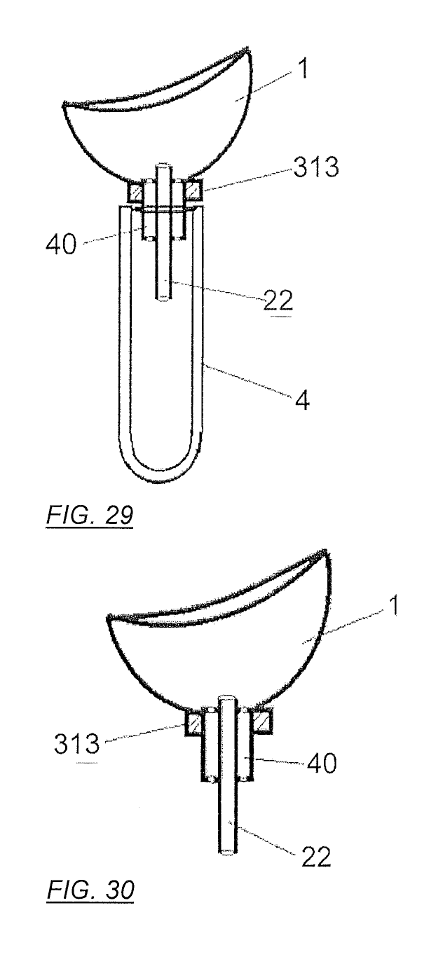

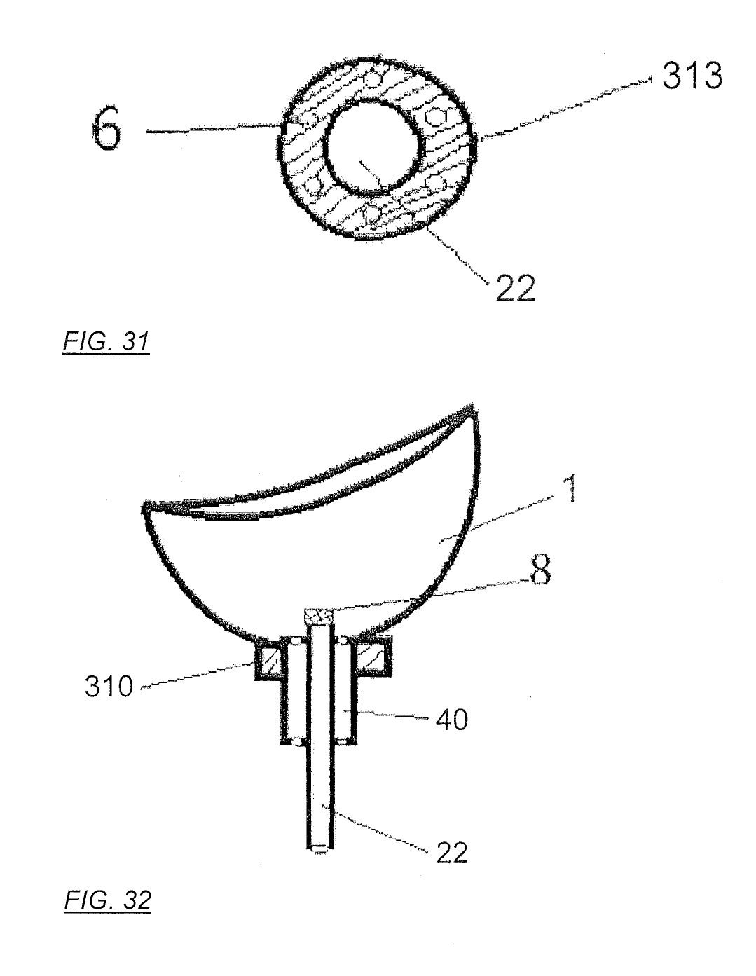

In yet another embodiment, as shown in FIGS. 29 and 30, in order to improve the convenience and stability of the urine collection and reduce the production cost, the utility model includes a urine collecting body for collecting urine and a urine collection tube 4 for storing urine. The urine collecting body includes a urine sampling funnel 1 and a connecting body 40 adapted to be connected to the urine collection tube 4, the connecting body 40 being fixed to the outer wall of the bottom end of the urine sampling funnel 1, the urine sampling funnel 1 being mounted by the connecting body 40 In the urine collection tube 4, there are provided a plurality of urine passages for urinating the urine and an exhaust passage for exhaust gas in the connecting body 40, the upper end of which is located at the upper end of the urine passage And the upper end of the exhaust passage is at least 5 mm above the upper end of the urine passage and the lower end of the exhaust passage is located below the lower end of the urine passage, and the urine collected by the urine sampling funnel 1 can pass through the urine passage and the exhaust passage Into the collection of urine collection tube 4.

Specifically, the urine collection tube 4 is an upper end opening and a lower end closed catheter, and the upper end of the urine collection tube 4 is connected to the urine collecting body, and the urine collected by the urine collecting body can be stored through the urine collection tube 4. The connecting body 40 is fixed to the urine sampling funnel 1, and the urine sampling funnel 1 has a funnel shape, and the urine sampling funnel 1 has a large opening for facilitating collection of urine. The urine sampling funnel 1 is attached to the upper end of the urine collection tube 4 through the connecting body 40, and the connecting body 40 is fitted in the urine collection tube 4, and the urine collected by the urine sampling funnel 1 enters the urine collection tube 4 through the urine passage.

In order to allow the urine to enter the urine collection tube 4, the exhaust passage is also provided, and when the urine enters the urine collection tube 4 through the urine passage, the gas in the urine collection tube 4 can be discharged through the exhaust passage; The upper end of the exhaust passage is located above the upper end of the urine passage, and generally, the difference in the height between the upper end of the exhaust passage and the upper end of the urine passage is at least 5 mm to ensure that the urine can be made more The low urine passage enters the urine collection tube 4, and the gas in the urine collection tube 4 is effectively exhausted through the exhaust passage. When the specific implementation, the exhaust channel and the urine channel between the height difference should not be too large, cannot enter the urine sampling funnel 1 too much to avoid affecting the normal urine collection, exhaust channel and the upper end of the upper part of the urine channel Height difference The specific size can be selected according to the need to set, to meet the needs of diversion and exhaust, here will not repeat them.

In addition, the lower end of the exhaust passage is located below the lower end of the urine passage, and the height of the urine collected in the urine collection tube 4 can be adjusted and controlled while improving the exhaust gas efficiency. In the specific implementation, the distance between the lower end of the exhaust passage and the lower end of the urine passage is not less than 1 cm. In addition, a water cutoff and exhaust film is provided at the upper end of the exhaust passage, and the gas is discharged by the water cutoff and the urine collected from the urine collection tube 4 is returned from the exhaust passage. Further, the space limiting block 313 for restricting the position of the connecting body 40 in the urine collection tube 4 is provided on the connecting body 40, and the stopper 3 is in contact with the outer wall of the bottom end of the urine sampling funnel 1.

In the embodiment, the space limiting block 313 is sleeved on the connecting body 40, the outer diameter of the space limiting block 313 than the inner diameter of the collecting duct 2, and the connecting body 40 is in the form of a column or a plug, and the connecting body 40 can be fitted In the urine collection tube 4 and in close contact with the urine collection tube 4. In the specific embodiment, the urine passage includes a guide pipe 6 provided in the connecting body 40, and the length of the flow guide pipe 6 can be made to coincide with the length of the connecting body 40. Of course, a plurality of diversion holes may be provided in the connecting body 40. A diversion passage is formed in the longitudinal direction of the connecting body 40, and the orifice penetrates the connecting body 40; the diversion hole 6 is uniformly distributed in the outer ring at the center of the connecting body 40. To ensure that the urine flows smoothly into the urine collection tube 4. The connection body 40 is in close contact with the urine collection tube 4 to prevent the urine in the urine sampling funnel 1 from leaking, and when the urine sampling funnel 1 is connected to the urine collection tube 4 by the connecting body 40, the urine sampling funnel 1 and the urine collection tube 4 can be used during the separation of the structure, reduce processing and transportation costs. In order to prevent the connecting 40 from being inserted too large in the urine collection tube 4, the space limiting block 313 is used to limit the connection.

Specifically, the exhaust passage includes an ventilation tube 22 provided in the connecting body 40, which is mounted in the connecting body 40 through the ventilation tube mounting hole 7 in the connecting body 40, and the ventilation tube 22 is located in the urine sampling funnel 1, and the other end of the ventilation tube 22 is communicated with the urine collection tube 4.

In the embodiment, the ventilation tube 22 is coaxially distributed with the connecting body 40, and the ventilation tube mounting hole 7 is located in the central region of the connecting body 40, and the ventilation tube mounting hole 7 penetrates the connecting body 40. As shown in FIG. 30, one end of the ventilation tube 22 is located in the urine sampling funnel 1, that is, the upper end portion of the ventilation tube 22 is higher than the upper end portion of the draft pipe 6 or the upper end portion of the ventilation tube 22 is higher than the upper end portion of the flow hole, When the manifold 6 is subjected to the urine guide, the ventilation tube 22 can achieve the purpose of rapid exhaust. Of course, the ventilation tube 22 may be located outside the axial center of the connecting body 40, and the ventilation tube 22 and the draft pipe 6 may be in the form of an interval distribution or other distribution as long as the effect of the introduction and the exhaust gas can be satisfied, Specifically not repeat them.

In order to prevent the urine from flowing through the ventilation tube 22 at the bottom of the urine hopper 1, the gas cannot be discharged, and the water cutoff film 8 may be provided at the top of the ventilation tube 22. As shown in FIG. 32, the urine does not enter the ventilation tube 22 from the upper portion of the ventilation tube 22 by the action of the water repelling gas permeable membrane 8 so as to ensure that the gas of the urine collecting pipe 2 can be discharged from the ventilation tube 22.

Further, a urine test strip is provided on the inner wall of the urine collection tube 4 for disposing the urine test strip in the urine state, and the urine test strip located in the urine test paper storage tank can be brought into contact with the urine stored in the urine collection tube 4.

In the embodiment, the urine test paper may be a PH test paper or other test paper for detecting urine, such as an early pregnancy test paper, a protein test paper, or the like. In general, the collection of urine collection tube 4 using a transparent tube, when the urine test paper placed in the urine test paper placed in the trough, the urine test paper can directly detect the urine, and directly observe the test results, improve the convenience of testing.

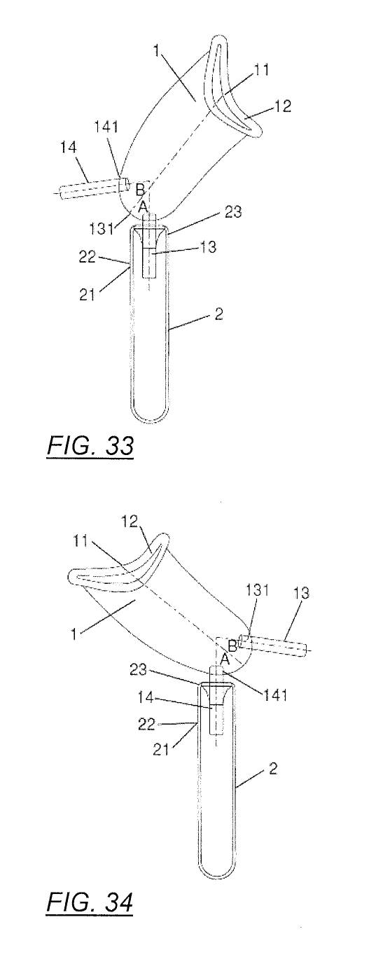

In yet another embodiment, referring to FIGS. 33 and 34, a filth-preventing urine sampling device includes a urine sampling funnel 1 that is disposed to cover the tip of the urethra to gather the urine, and that prevents urine from overflowing and contaminating the patient's hand, and a quantitative backflow prevention conduit 2 used with the urine sampling funnel 1 to collect the urine while preventing both overflow of urine and urine backflow. The urine sampling funnel 1 has an curved opening 11 that is shaped to match with the external shape of the female perineum, and the bottom of the urine sampling funnel 1 is provided with an outlet tube 13, where the urine is discharged out from the inside of the urine sampling funnel 1. An opening 21 is disposed on the upper wall part of the quantitative backflow prevention conduit 2, and within the opening 21 a water blocking/gas permeable membrane 22 is provided. Further, on the quantitative backflow conduit 2, the upper interior part of the water blocking gas permeable membrane 22 is provided with a one-directional soft membrane 23.

During male use, one inserts the penis into the curved opening 11 of the urine sampling funnel 1, and the urine flows along the inner wall of the urine sampling funnel 1, to the bottom of the urine sampling funnel 1 and out of the outlet tube 13, and eventually into the inner lumen of the quantitative backflow prevention conduit 2. During female use, the usage method is slightly different from male use, with the curved opening 11 of the urine sampling funnel 1 covering matching the shape of the female perineum and covering the perineum, urine sampling funnel 1 arcuate inner wall of the curved opening 11 urine sampling funnel 1 flowing in the perineum, the urine will in urine, the final outlet along the bottom of the urine sampling funnel 1, ultimately into the cavity 2 of the quantitative backflow conduit.

As the urine backflow prevention quantitatively into the cavity 2 of the catheter, the catheter quantitative backflow gas discharge chamber 2 from the gas permeable membrane of the water blocking backflow quantitative upper portion 22 of the catheter 2, so that the urine backflow prevention pipe quantitative 2 until the remaining water blocking permeable membrane cavity 22 is flooded. Thereafter, the one-way membrane 23 pairs of a flexible inlet tube 13 surrounding the sealing effect, preventing backflow quantitative gas duct lumen 2 is not unidirectional soft film 23 and the outer wall of the pipe-like discharge gap 13, while the water blocking gas permeable membrane 22 is not submerged discharge, urine backflow into the catheter lumen quantified 2 is stopped, the sample extraction tube 13, taking the end of the urine.

What is needed is a water-blocking position of the upper edge of the gas permeable membrane 4 directly determines the exact kind of liquid tube 13 can quantify the amount of backflow into the catheter lumen 2, the higher the upper edge of the gas permeable membrane water blocking position 4, to collect the more liquid the lower position, the less liquid can be collected. When pulled out of the sample tube 13, since the sample tube 13 occupied by the space becomes smaller, the fluid level is slightly decreased, the gas permeable membrane 4 uppermost water-blocking fluid leakage violent plane, but the sample liquid in the tube lumen 13 continues downstream complement lowered liquid surface, the gas permeable membrane is still in the water blocking flooding the upper edge of the suspension 4, is still accurate quantification.

The sidewall of the catheter, the gas permeable membrane preferably only in the water-blocking capacity of the uppermost end 4 and the capacity value of the mark, the uppermost mark 4 can be determined quantitatively by reading the water blocking gas permeable membrane 2 taken backflow catheter fluid volume, fluid conduits of different colors in different amounts taken tick preferably, convenient for the user to identify pick.

The curved opening 11 of the urine sampling funnel 1 is provided with an arched shape that matches the female perineum of urine taken for increasing the sealing skirt 12. Sealing skirt 12 may be disposed in a gap between the curved opening 11 and the arcuate outer edge of the curved opening 11, can increase tightness when women taking urine from the urine to reduce the arcuate edge of the curved opening 11 of the inner arcuate opening, it may be provided the possibility of spills.

The bottom 13 of the urine sampling funnel 1 of the sample tube into the urine within the urine port 131 for the urine sampling funnel 1's surface. The vicinity of the sample tube 13 is provided for preventing the overflow of urine bypass pipe 14, bypass pipe 14 and the urine sampling funnel 1 communication port at the bottom split urine sampling funnel 1 141 the inner surface of the urine sampling funnel 1.

During male use, the shunt tubes 14 is provided when the sample tube 13 into quantitative backflow conduit 2, a quantitative urine backflow prevention filled catheter 2 in the urine sample after the memory I funnel plot. When urine in the urine diversion port 141 exceeds the level of the shunt tube 14, the urine will be discharged out of the multi-lumen from the shunt tube 14, to avoid the urine accumulated in the memory continues to the urine sampling funnel 1, even by the curved opening 11 of the urine sampling funnel 1, causing the patient to take the skin of the perineum, or urine contaminated the urine sampling funnel 1 held the hand portion. Meanwhile, urine diversion port 141 bypass pipe 14 is higher than the inner surface of the urine sampling funnel 1, can be reduced or avoided split urine from the urine inlet tube 14 when the shunt 141 is less direct discharge, is not conducive to urine sampling.

During female use, as shown in FIG. 34, urine sampling funnel 1 is shaped to have different angles, 13 and change the position of the bypass pipe 14 using the specific sample tube, sample tube resorted shunt tubes 14 and 13 function alternately. Shunt tube 14 is lower than the position of the sample tube 13, to assume a urine sample tube 13 is discharged to the dosing backflow conduit 2 features. Sample tube 13 out of position higher than the shunt tubes 14, 14 assume the excessive discharge of urine to avoid overflow of urine function shunt tube. In order to better resorted sample tube 13 assume the function of the shunt tube 14, the urine into the sample tube 13 within the opening 131 for the urine sampling funnel 1's surface. In specific implementation, urine urinary diversion port 141 and port 131 into the urine sampling funnel height above the surface is not too high I, preferably 0.5 cm-lcm.

The lumen of the urine sampling funnel 1 includes an arcuate central axis, the axis of the curved opening 11 in the sample tube axis A 13 is angled 30-60 degrees angle. The shunt tubes 14 in the sample tube 13 with the axis of the central axis at an angle of 30-85 degrees angle B.

The urine sampling funnel is designed to have a curved shape, so that the urine out of the urethra in the direction and orientation of the sample tube 13 and bypass tube 14 form a cross, can avoid the use of urine from the urine of male shunt tube splitter 14 spill port 141, while avoiding the use of urine from women when the sample tube 13 into the urinary spill port 131.

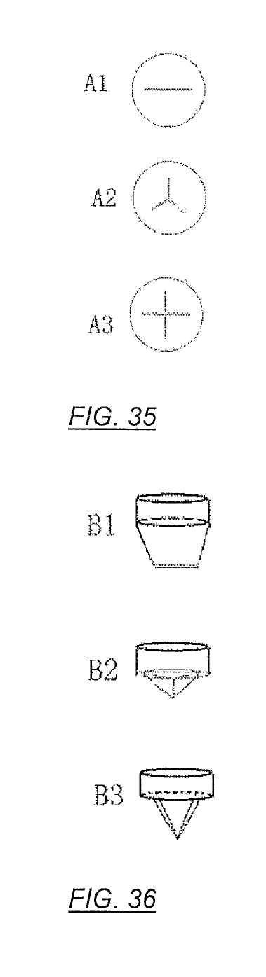

FIGS. 35 and 36, the flexible membrane 23 comprises a bowl-shaped one-way membrane, the membrane bowl diameter mouth portion 2 of the catheter lumen quantitative backflow match, the bowl bottom film is provided fissures, including linear slit diaphragm A1, A2, and pointed star cross slit diaphragm slit diaphragm A3. The one-way valve further comprises a flexible film 2, an upper mouth portion to the diameter of the valve is provided with a lumen quantitative backflow matching annular conduit 2 near the lower portion of the valve flap arranged close to the edge, comprising two valves B1, B2 and three valve four valve B3.

Such flexible film 13 provided on the sample tube 14 into the bypass pipe or quantitative backflow conduit when the valve clearance gap or opening 2, passed; quantified backflow prevention outflow conduit 2 when the liquid has tendency to gap or gaps valve flexible film around the hydraulic deformation under tight plugging into the quantitative backflow duct 2 around the sample tube 13 or the shunt tubes 14 and the check clearance soft film 23, a liquid reflux is not, while the backflow prevention pipe quantitative urine sampling time gas in the gap 2 thereby cannot be discharged. Quantitative extraction backflow into the catheter 2 or the sample tube 13 when the shunt tubes 14, flexible deformation of the film cannot be pulled out against a large force, the sample tube 13 or the shunt tubes 14 can be smoothly pulled in.

The active closure opening 21 is provided, the openings 21 can be blocked. At the end of the sample, using the closure plug the opening 21, avoiding too long time, urine may permeate through the water cut-permeable membrane 22 to increase the safety in use.

By having the urine sampling funnel 1 envelope the periphery of the urethra of men and women while they urinate, urine is less likely to spill, shunt tubes to avoid contamination, the upper portion of the water blocking quantitative backflow conduit tube-like gas permeable membrane resorted the urine can be smoothly discharged urine was sampled and quantified, the one-way soft film chamber to avoid urine outflow, simple, safe, reliable, practical.

In yet another embodiment, an antifouling take liquid from the discharge tube 1 is provided, including a soft film were washed with a unidirectional catheter body is provided at an upper portion 1 and lumen catheter body 2, the catheter body at the upper wall 3 is provided at least one opening, hole disposed the water blocking seal 4 the gas permeable membrane.

When the specific use, take liquid into the tube 2 by way flexible film into the cavity of the catheter body I, 2-way soft film wrapped around the tube so that a tapping a tapping tube wall cavity remains sealed and the catheter body, the catheter body gas chamber I cannot get through the check flexible film wrapped around the liquid pipe and a liquid pipe taking the gap between the discharge. When taking the catheter body fluid gas chamber I through the upper wall of the opening can be gas-permeable membrane disposed inside the water blocking 34 of the catheter body 1 is discharged, so that body fluids can smoothly into the cavity through the catheter body I taking catheter. When the liquid enters the lumen of the catheter body I, the fluid level rises gradually, when the liquid surface of the water blocking flooding the top gas permeable membrane 4, the catheter lumen cannot continue to discharge the gas, take liquid suspension, the upper edge of the gas permeable membrane 4 of the water blocking position taken directly decide precisely the amount of liquid to enter the liquid pipe catheter body cavity I, the higher the water blocking position of the gas permeable membrane 4 of the upper edge, the more liquid can be collected in the lower position, the less liquid can be collected. When taking liquid pipe pulled, due to the space occupied by taking small liquid pipe, a fluid level slightly decreased, the gas permeable membrane 4 uppermost water-blocking fluid leakage violent plane, but the liquid chamber continues to take liquid downflow tube falling replenisher plane, the still plane when submerged edge of the suspension liquid still accurate quantification.

The sidewall of the catheter of the present vitro tick capacity, the gas permeable membrane preferably only in the water-blocking capacity of the uppermost end 4 and the capacity value of the mark, the mark 4 uppermost present invention can be determined by reading the water blocking catheter permeable membrane I taken body fluid volume, fluid conduits of different colors in different amounts taken tick preferably, convenient for the user to identify pick.

The flexible film comprises a bowl-shaped one-way membrane, the membrane bowl mouth diameter matching lumen of the catheter body, the bottom of the bowl-shaped film disposed fracture, a linear fracture membrane comprising A1, A2, and pointed star cross slit diaphragm slit diaphragm A3. This flexible membrane disposed cracks open when taken into the pipette, passed; the catheter body I with a liquid outflow when the tendency to deformation of the flexible film around the cracks in hydraulic, tight plugging the gap around the catheter taken, can liquid reflux. While taking liquid tube pulled out, soft membrane deformation cannot be pulled out against a larger force, to take liquid pipe smoothly pulled out.

The one-way valve comprises a flexible membrane, a valve provided to an upper opening diameter of the catheter body of this annular chamber matching, near the lower portion of the valve flap arranged close to the edge, comprising two valves B1, B2 and three four-valve valve B3. This valve is more common in stamp card (laparoscopic puncture), a not repeat them here, using the principles above.

The active closure opening 3 is provided, can plug the opening after the sample 3o, 3 using the closure opening blocked, to avoid too long, the liquid may permeate through the water cut-off the gas permeable membrane 4, the increased use of security.

The above structure can prevent an upper portion of the asymmetric membrane of the catheter body backflow of the liquid; gas permeable membrane allows the water blocking gas discharge conduit body, smooth filling liquid sampling tube; water blocking position enables the gas permeable membrane disposed to note the exact amount of liquid added.

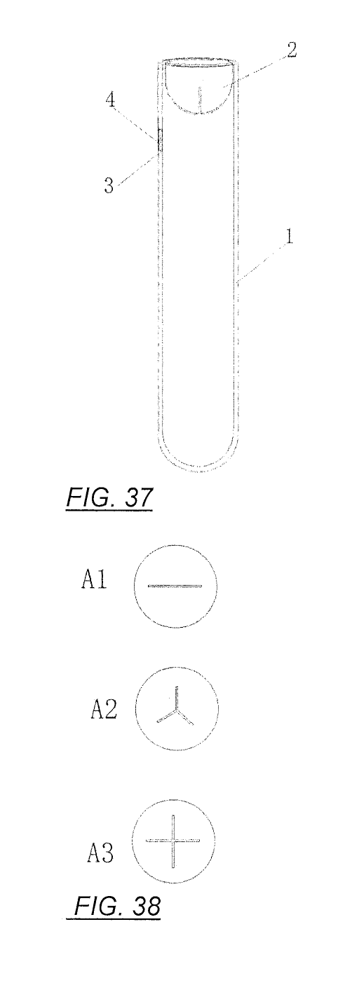

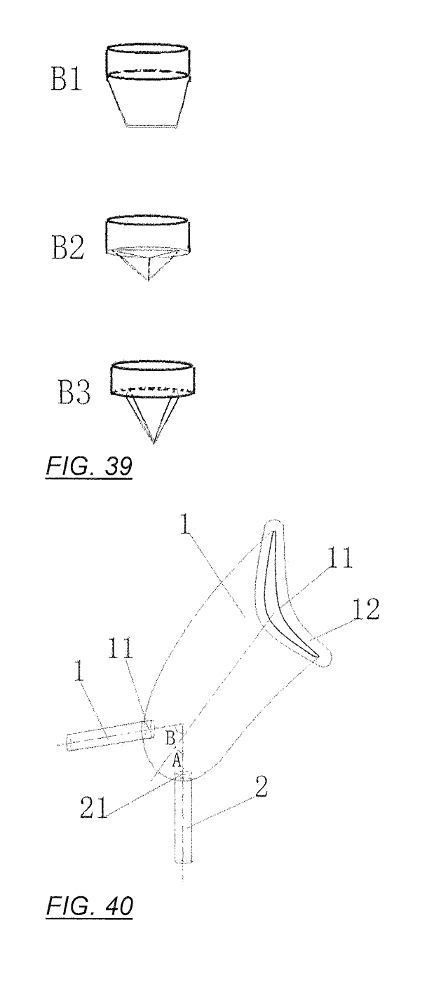

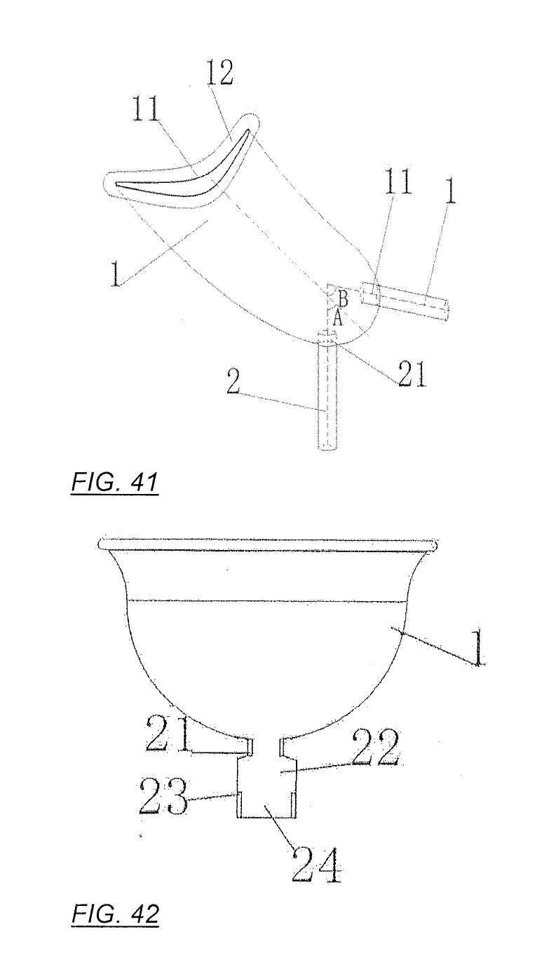

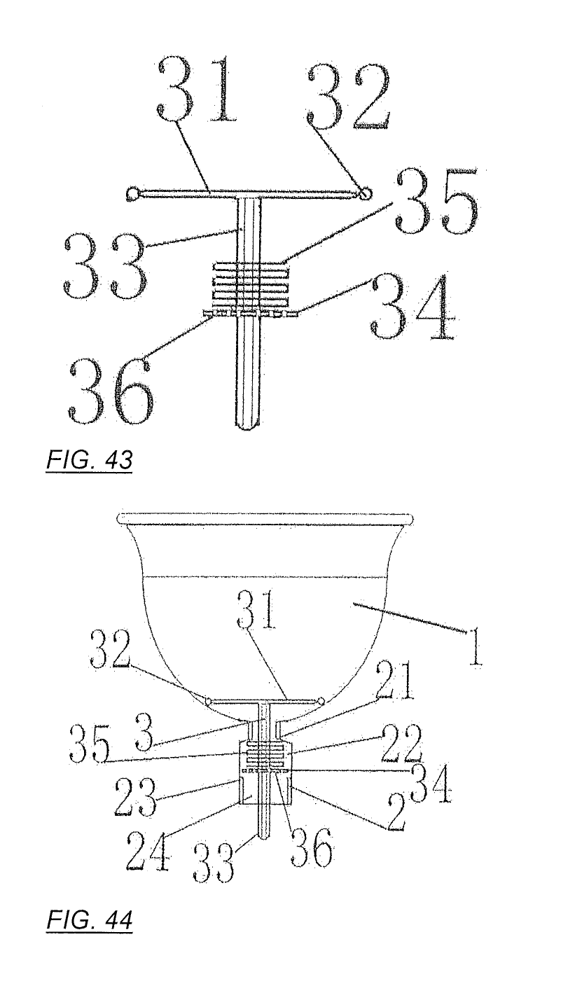

In yet another embodiment, referring to FIG. 37 and a funnel unisex urine sampling type, includes a sleeve connected to opposite filled urethra urine in the funnel and the funnel 1 pot bottom of the funnel lumen 2 communication for drainage of urine drainage tube 2. The funnel mouth 1 to match the shape of the female perineum arcuate opening 11, the inner diameter of the funnel kettle I is greater than 3 cm.

During female use, only the female perineum arcuate outer shape matching an opening 11 in the cover ring perineum, the urine will be along the funnel wall I flowing along the funnel final pot bottom 2 I drainage discharge tube. When men, only the penis into the hopper 11 from the arcuate opening cavity of the pot I, also in urine funnel flow pot I pot wall, the pot ultimately along the drainage pipe at the bottom of the hopper 2 is discharged I. Connected with the urine container underneath the drainage pipe 2 to get a urine sample.

The funnel opening 11 is provided with an arcuate female perineum match the shape of urine taken for increasing the sealing skirt 12 clothes. Skirt coating 12 may be disposed in a gap between the opening 11 and the arcuate outer edge of the skin 11, can increase tightness when women taking urine from the urine to reduce the arcuate edge of the opening 11 of the inner arcuate opening, it may be provided the possibility of spills.

The pot bottom of the funnel 1 into the urine drainage tube 2 is provided near the opening 21 for preventing the overflow of urine shunt tube 3, the shunt tubes 3 communicates with pot I funnel lumen, the urine at the bottom of the funnel split pot 1, high port 31 for the inner surface of the funnel pot I.

The purpose of the shunt tubes 3 provided that when the urine drainage tube 2 placed in a sealed sampling vessel, after the urine container filled with pot I funnel plot in the memory. When urine spill over port 31 when the horizontal tubes 3, a plurality of urine from the shunt tube will discharge chamber 3, to avoid continued funnel urine volume pot I memory, and even the pot I arcuate hopper overflow opening 11, causing the patient to take the perineal skin or the hand holding the funnel pot I was contaminated urine.

In the\ vent 3 the shunt tube 31 higher than the inner surface of the funnel pot I may be reduced or avoided from the manifold tube 3 when the bypass port 31 is directly discharged urine less, is not conducive to urine sampling.

According to the above structure, during male use, the patient using a funnel pot I arcuate opening sideward direction of the patient 11, the drainage tube the funnel 2 bottommost pot 1, the bypass tube 3 is located slightly above the bottom region of the funnel pot I drainage tube 2 eventually play take urine and prevent urine from overflowing urine diversion function.

As shown in FIG. 38, when the present invention is a female patient using a funnel pot I arcuate opening 11 upward at this time, the shunt tubes 3 I lowermost pot of the funnel functions as a urine sample extraction, drainage bear function pipe. The draft tube 2 positioned slightly above the bottom region of the funnel pot I shunt tube 3, assume the function of the shunt, and prevent urine overflowing. Eventually, shunt tubes 2 and 3 positions interchanged draft tube, alternately function, also play take urine and prevent urine from overflowing urine diversion function.

In order to prevent the urine sample is less when the urine drainage tube inlet 21 of the discharge port 2 is not conducive to urine sampling, the bottom of the funnel kettle I into the urine drainage tube 2 at the bottom of the hopper inlet 21 of the pot I when women use the high for the funnel I pot surface, when there are fewer urine into the urine inlet below the plane 21 of the draft tube, so as to avoid loss of urine less.

The pot funnel 1 lumen includes an arcuate central axis, the axis of the arcuate opening 11 in the draft tube axis 2 records the angle A of 30 to 60 degrees. Meanwhile, the shunt tube 3 and the axis 2 records draft tube axis angle B from 30 to 85 degrees.

The arcuate funnel pot I set, the direction 2 so that the urine tube 3 and the shunt direction and urethral drainage tube form a cross, can avoid the use of male urine from the urine directly shunt tube 3 spill port 31, while women avoid the use of urine spilled directly from the mouth into the urine drainage tube 2 of 21.

The funnel 1 described above can be used in matching male and female patients, the drainage pipe 2 and the bottom 3 a shunt tube location and function can substitute for each other when both men and women, the structure is simple and practical.

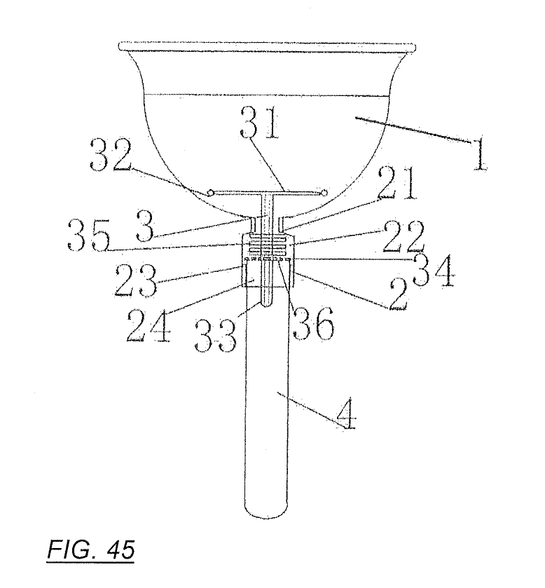

As shown in FIGS. 44 and 45 a urine sampling valve controlled funnel, includes a container holding the contact for receiving the urine by the inverted flared portion 1, the vessel I is provided in the lower end of the urine can then take urinary catheters and temporary catheter seal 4 is fixed and the fixing sleeve 2 is provided in the conduit for controlling the fixing sleeve 2 contact the urine receptacle cavity I a lower channel opening and closing the flow of urine touches the seal spool 3.

When receiving the urine, the urine receptacle contact section I flare up, the patient is on the urethra opening, a rear urine urethra bell mouth shape to reduce urine splash, spill and to avoid splashing of urine pollution patient's finger. After the urine into the urine receptacle contact cavity 1, the urine accumulates to the bottom of the vessel I is connected, through the outflow lumen catheter fixing sleeve 2 taken urinary catheters into the lumen 4. In the cavity of the catheter fixing sleeve 2 is provided touches seal spool 3, the spool 3 touches the front seal untouched, the valve element 3 touches the sealing blocked urine the urine outlet port connected to the bottom of the container I, lumen catheter fixing sleeve 2 is closed, receiving urine I urine receptacle cavity cannot be discharged. 4 when taken urinary catheters inserted catheter fixing sleeve 2, the holder 4 takes urinary catheters touch seal spool 3, so that the urine outlet opening connected to the bottom of the container I, urinary catheter lumen patency sleeve 2 is fixed, then the urine receptacle I urine lumen catheter can be smoothly flows into the lumen of the fixed sleeve 2 taken urinary catheter 4 lumen. After completion of taking of urine, urinary catheters 4 detachment taking simultaneously touches recovery seal spool 3, the spool 3 again touches the sealing contact urinary blockage of urine flow outlet at the bottom of the container 1, the lumen of the catheter fixing sleeve 2 is closed, then the urine receptacle chamber I the urine cannot be discharged, the patient hands to avoid urine contamination.

The conduit sleeve 3 fixed conduit 4 and the upper end of the outer wall of urine taken to match the urinary catheter takes the inner wall of the catheter 4 opposite sets of fixed sleeve 3 and the upper portion of the upper end of the outer wall 4 taken urinary catheters in sealing contact with the sleeve 3 fixed to the catheter lumen.

The effect of: fixing sleeve sleeved conduit 4 into conduit can take the temporary fixing of the urine 3, to avoid falling off; when taking urine from the urine into the conduit 4, taken urinary catheter 4 when filled with urine, the urine also catheter securement sleeve 3 and will not take the overflow of urine by contact portion 4 of the catheter.

The urine receptacle contact comprising a contact urinary I funnel, then the bag. Taken when the urine is not much difference, but then the pouch bag soft, easy to fold storage, reducing the volume, reduce transportation costs. If set to take the bag, it should be provided for withholding the ring member with the expansion opening, ease of use.

The conduit sleeve 3 is preferably fixed to the tapered opening to facilitate the taking of urine entering the catheter 4 opposite the upper end of the conduit sleeve 3 fixed to the sleeve, to reduce the cumbersome alignment operation.

The valve element 3 includes an upper seal touch appearances, and the urine container 31 I-compatible sealing disc, an outer edge of the sealing plate 31 is provided a flexible sealing ring 32. The side of the sealing plate 31 corresponding to the center position of the catheter 2 is provided a fixed guide sleeve core 33, the core guide 33 is provided to touch the lower fixing member 34 touches the body 34 is provided on a plurality of through-holes 36 below. The outer guide 33 between the core 31 and sealing disk 34 touches the spring sleeve 35 is provided.

The sealing plate 31 for sealing an outlet connected to the lower portion I of the urine receptacle, and then its appearance consistent lower interior section of the container of urine I, preferably circular set. The flexible sealing ring 32 and the edge of the disc 31 match the shape, to increase the tightness of the sealing plate 31 and contact the inner wall of the lumen of the lower portion I of the urine receptacle is provided. Core guide catheter 33 provided on the fixed bit position of the center sleeve 2, to ensure that the sealing plate 31 and flexible seal 32 and then the correct alignment of the inner wall of the lower portion I of the urine receptacle. Core guide surface 33 is preferably provided with a plurality of separate guide grooves distributed along the major axis, while the guide, not occupied by the liquid guide grooves can play the role of an exhaust groove, the exhaust gas conduit 4 take the urine, urine liquid can successfully enter take 4 urinary catheters.

The spring 35 provides power to touch the seal spool 3, a mild compression ring 32 can press the flexible disc 31 of the edge seal 35 against the spring force of the contact of the urine outlet of the container lower portion I, then clogging the urine flow outlet in the bottom of the container I. Severe compressed, flexible ring 31 and the sealing disc 32 from the rear edge of the lightly compressed by the force of the spring 35 connected to the outlet of the urine receptacle inner wall of the lower portion I, then I the urine container bottom outlet opening of the flow of urine, the urine can flow through the part surface of the core guide grooves 33 disposed within the conduit 4 flows into the urine taken while, to take the gas discharge urinary catheters 4 via the other guide groove.

Lower fixing member 34 is disposed below the plurality of through-holes 36 purpose is to avoid taking the urine during use of the catheter 4 at the upper opening at the top holding member 34 side surface of touch, urinary catheters to avoid taking opening 4 is blocked, resulting in not take urine or urinary efficiency.

As shown in FIG. 42, the catheter fixing sleeve 2 is provided for the middle spring chamber accommodating the spring 22, the spring chamber 22 upper opening portion 21 can be provided on a first limiting edge of the stopper spring 35, the spring the lower cavity portion 22 can be provided on the second stop 34 touches the lower edge of the stopper body 23. The catheter sleeve 2 fixed in a lower portion of the second stopper member 23 is provided and can take the upper temporary seal 4 urinary catheters catheter lock chamber 24 is fixed.

The first limiting member 21 can prevent an upper restoring spring 35, the spring 35 remains slightly compressed state prior to use, the flexible seal sealing disc 31 edge 32 contact the inner wall pressing the lower outlet of the urine receptacle 1, increase tightness. The second stopper member 23 can be touched to the stopper 34, and thus the lower edge of the stopper spring 35 to prevent spring 35 through the lower recovery, the loss of the compression power. Fixing the catheter lumen 24 to be taken and urinary catheters 4 upper outer sealing contact to maintain sealing performance, meaning as above mentioned.

In FIG. 45, a schematic view of the device in a used state, in order to achieve the above results, must be done: the spring 35 is located within the spring chamber 22, spring 35 is greater than the length of the stopper body 21 and a first the distance between the spring chamber 22 of the second stopper member 23. The spring chamber 22 is greater than the outer diameter of the lumen 34 of the touch body, the outer diameter of the spring member 34 is smaller than the diameter 35 touches the outer diameter of body 34 is greater than the inner diameter of the urinary catheter takes 4.

The spring length is greater than the distance between the spring chamber of the first stopper 2322 and the second stopper member 21, in order to ensure that the spring 35 is always in a compressed state, function to provide power for subsequent use. Lumen 22 is greater than the outer diameter of the spring chamber 34 of the body touches to the touch body 34 fitted within the spring chamber 22. Similarly, only the spring 35 is smaller than the diameter of the body 34 touches, in order to make effective the top spring holder 35 touches the body 34, does not emerge from the body 34 touch. Only the outer diameter of the body 34 touches an inner diameter larger than the urinary catheter taken 4, whichever urinary catheters into the catheter 4 within the chamber 24 can be effectively fixed top holding member 34 touches, while avoiding touching the body 34 sleeved conduit 4 into the lumen of the urine was taken cannot touch the seal spool 3 to extract urine fail.

FIG. 45 is a schematic view of urine sampling valve controlled funnel in use to extract urine, in which working principle is: Take urinary collector 4, with holder 34 touching the spring member 35, which is heavily compressed and touches the seal spool 3 is raised, the flexible ring 32 lifted off the urine receivable I lower outlet of the container inner wall, the urine clearance by the contact of the urine receptacle I and the lower outlet 33 flows to the guide core within the spring chamber 22. At the same time, also touch the body 34 is lifted up from the second stopper member 23, the holes provided in the portion touched by the body of urine 34 within the chamber 36 to flow to the spring 22 to take urine from the conduit 4. At the same time, the other apertures 4 into the gas taken through the urinary catheter 34 disposed on touch 36 is discharged into the spring chamber 22, and is discharged through the gap outlet and a lower portion of the vessel I is connected to the urine guide core 33. When completed take the urine. 4 taken detachment urinary catheters moment, under the action of spring 35, seal spool 3 drops touch, flexible sealing ring 32 pressed against the lower outlet connected to the inner wall of the urine container I, the residual urine cannot be discharged to avoid contamination.

Based on the above, the urine receptacle 1 contacts to reduce contact of urine splashing urine, the catheter 2 is temporarily fixed fixing sleeve 4 takes urinary catheters, urinary catheters taken into four holding seal spool 3 touches so that the urine flows into the gas and discharge passage open, to take complete urinary; taken after detachment urinary catheters 4 are simultaneously touched reset seal spool 3, and an exhaust passage of urine taken off. To avoid the risk of contamination of health care, is easy to use, has simple structure, and is worthy of promotion.

### Non-limiting Discussion of Terminology

The headings (such as “Introduction” and “Summary”) and sub-headings used herein are intended only for general organization of topics within the present disclosure, and are not intended to limit the disclosure of the technology or any aspect thereof. In particular, subject matter disclosed in the “Introduction” may include novel technology and may not constitute a recitation of prior art. Subject matter disclosed in the “Summary” is not an exhaustive or complete disclosure of the entire scope of the technology or any embodiments thereof.

The description and specific examples, while indicating embodiments of the technology, are intended for purposes of illustration only and are not intended to limit the scope of the technology. Equivalent changes, modifications and variations of specific embodiments, materials, compositions and methods may be made within the scope of the present technology, with substantially similar results. Moreover, recitation of multiple embodiments having stated features is not intended to exclude other embodiments having additional features, or other embodiments incorporating different combinations of the stated features. For example, a component which may be A, B, C, D or E, or combinations thereof, may also be defined, in some embodiments, to be A, B, C, or combinations thereof.

Specific examples are provided for illustrative purposes of how to make and use the compositions and methods of this technology and, unless explicitly stated otherwise, are not intended to be a representation that given embodiments of this technology have, or have not, been made or tested.

As used herein, the words “prefer” or “preferable” refer to embodiments of the technology that afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the technology.

As used herein, the word “include,” and its variants, is intended to be non-limiting, such that recitation of items in a list is not to the exclusion of other like items that may also be useful in the materials, compositions, devices, and methods of this technology. Similarly, the terms “can” and “may” and their variants are intended to be non-limiting, such that recitation that an embodiment can or may comprise certain elements or features does not exclude other embodiments of the present technology that do not contain those elements or features.

Although the open-ended term “comprising,” as a synonym of non-restrictive terms such as including, containing, or having, is used herein to describe and claim embodiments of the present technology, embodiments may alternatively be described using more limiting terms such as “consisting of” or “consisting essentially of.” Thus, for any given embodiment reciting materials, components or process steps, the present technology also specifically includes embodiments consisting of, or consisting essentially of, such materials, components or processes excluding additional materials, components or processes (for consisting of) and excluding additional materials, components or processes affecting the significant properties of the embodiment (for consisting essentially of), even though such additional materials, components or processes are not explicitly recited in this application. For example, recitation of a composition or process reciting elements A, B and C specifically envisions embodiments consisting of, and consisting essentially of, A, B and C, excluding an element D that may be recited in the art, even though element D is not explicitly described as being excluded herein. Further, as used herein the term “consisting essentially of” recited materials or components envisions embodiments “consisting of” the recited materials or components.

“A” and “an” as used herein indicate “at least one” of the item is present; a plurality of such items may be present, when possible.

When an element or layer is referred to as being “on”, “engaged to”, “connected to” or “coupled to” another element or layer, it may be directly on, engaged, connected or coupled to the other element or layer, or intervening elements or layers may be present. In contrast, when an element is referred to as being “directly on,” “directly engaged to”, “directly connected to” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Spatially relative terms, such as “inner,” “outer,” “beneath”, “below”, “lower”, “above”, “upper” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

What is claimed is:

1. A urine sample device, comprising:
a funnel for receiving urine, the funnel in connection with a guide tube at a lower portion of the funnel;
a urine collection tube for collecting the urine that flows from the funnel and through the guide tube; and
a guide tube fixation member located at a lower portion of the funnel and an upper portion of the guide tube along an outer periphery of the guide tube, the guide tube fixation member connected to the urine collection tube at an upper portion of the urine guide tube; and
a vent located inside the guide tube fixation member.

2. The urine sample device according to claim 1, wherein the guide tube fixation member further includes a spacer located at an upper portion of the guide tube fixation member, the spacer fixable to an upper region of the urine collection tube.

3. The urine sample device according to claim 1, wherein the vent further includes a ventilation tube in sealed communication with the vent.

4. The urine sample device according to claim 1, wherein the vent provided in the guide tube fixation member is located near the funnel.

5. A self-ventilating urine sampling funnel, comprising:
a bell section;
a guide tube connected to a bottom portion of the bell section;
an access for containing urine inverted bell section, provided at the lower end of the guide tube lumen and a bell flare portion in communication with and disposed in the guide tube into the outer wall of the insert taken for urinary drainage catheter and that can take urinary catheters gas is fixed within a guide tube fixation member, wherein:
the guide tube fixation member and the lower part takes urinary catheters is fixed to the upper end of the outer wall of the catheter match chamber, near the bell mouth portion of the catheter lock chamber side end seal catheter lock chamber away from the bell section of the side of the open end; the catheter lock chamber in communication with the sealed end exhaust pipe and pipe fixing side chamber.

6. The self-ventilating urine sampling funnel according to claim 1, wherein the ventilation tube is located near the closed end of the guide tube fixation chamber.

7. The self-ventilating urine sampling funnel according to claim 1, wherein the ventilation tube is located near a closed end that is fixed to a side wall of the guide tube fixation chamber.

8. The self-ventilating urine sampling funnel according to claim 7, wherein
the closed end of the guide tube fixation chamber is located at a side end portion of the space limiting block, and
a space limit point of the space limiting block is not lower than a gas inlet of the ventilation tube.

9. A urine sampling bag, comprising;
a urine sampling bag body for collecting urine, the sampling bag disposed at the upper end of a urine collection tube fixed to a guide tube fixation member; and
a guide tube and a ventilation tube located within the guide tube fixation member in communication with the sampling bag.

10. The urine sampling bag according to claim 9, wherein
the guide tube is fixed to the catheter body remote from the sampling bag than the length of one side of the same side of the ventilation tube, and
the length of the ventilation tube in a guide tube fixation side of the sampling bag length is longer than that of the guide tube on the same side.

11. The urine sampling bag according to claim 9, wherein the urine sampling bag body is provided elastic ring at an opening portion.

12. The urine sampling bag according to claim 9, wherein
the sampling bag provided at the bottom with a guide tube inlet.

13. The urine sampling bag according to claim 9, wherein the guide tube fixation member includes an inner cavity and a guide tube fixation member includes a holding sleeve, the outer diameter of the outer wall of the urine collection tube meets the holding sleeve.
